(19) United States
(12) Patent Application Publication
(10) Pub. No.: US 2025/0044284 A1
(43) Pub. Date: Feb. 6, 2025

(54) MATERIALS AND METHODS FOR ENHANCED DETECTION AND SCREENING OF MOLECULES OF THERAPEUTIC POTENTIAL

(71) Applicant: Janssen Biotech, Inc, Horsham, PA (US)

(72) Inventors: Vinicius CARREIRA, San Diego, CA (US); Mathieu MARELLA, San Diego, NJ (US)

(21) Appl. No.: 18/572,451

(22) PCT Filed: Jun. 23, 2022
(86) PCT No.: PCT/IB2022/055835

§ 371 Date: Dec. 20, 2023

Related U.S. Application Data
(60) Provisional application No. 63/214,177, filed on Jun. 23, 2021.

(30) Foreign Application Priority Data
Jun. 23, 2022 (WO) PCT/IB2022/055835

(51) Int. Cl. G01N 33/53; G01N 1/30; G01N 1/36; G01N 1/44; G01N 33/68

(52) U.S. Cl. CPC G01N 33/5308; G01N 1/30; G01N 1/36; G01N 1/44; G01N 33/68; G01N 2001/305

(57) ABSTRACT

Provided herein are methods of detecting the presence of a molecule in a sample, such as a bodily fluid or tissue of a patient.

### CROSS-REFERENCE TO RELATED APPLICATIONS

This application claims the benefit of U.S. Ser. No. 63/214,177 filed Jun. 23, 2021, which is incorporated herein by reference in its entirety.

### FIELD

Provided herein are methods of detecting the presence of a molecule in a sample, such as a bodily fluid or tissue of a patient.

### SUMMARY

In one aspect, provided herein is a method of detecting a molecule in a sample, comprising: obtaining cells from a sample; treating the cells with a fixative; paraffin embedding the fixed cells; deparaffinizing and suspending the cells to obtain a single cell suspension; contacting the suspended cells with a first detection agent that binds at least one molecule of the suspended cells; contacting the cells bound to the first detection agent with a second detection agent; and detecting the presence of the second detection agent bound to the cells of the sample; wherein detection above background of an amount of the second detection agent bound to the sample indicates the presence of at least one molecule in the sample.

In some embodiments, the molecule is a nucleic acid or a protein. In some embodiments, the nucleic acid is RNA. In some embodiments, the nucleic acid is DNA.

In some embodiments, the method further removing unbound cells after contacting the suspended cells with a first detection agent. In some embodiments, the method further comprising removing the unbound second detection agent after contacting the cells bound to the first detection agent with a second detection agent.

In some embodiments, the fixative is selected from a group comprising formaldehyde, paraformaldehyde, glutaraldehyde or neutral buffered formalin. In some embodiments, the fixative is neutral buffered formalin. In some embodiments, the neutral buffered formalin is 10% neutral buffered formalin.

In some embodiments, the step of treating the cells with the fixative lasts for about about 30 minutes to 60 minutes, 1 hour to 2 hours, 2 hours to 3 hours, 3 hours to 4 hours, 4 hours to 5 hours, 5 hours to 6 hours, 6 hours to 7 hours, 7 hours to 8 hours, 8 hours to 9 hours, 9 hours to 10 hours, 10 hours to 11 hours, 11 hours to 12 hours, 12 hours to 13 hours, 13 hours to 14 hours, 14 hours to 15 hours, 15 hours to 16 hours, 16 hours to 17 hours, 17 hours to 18 hours, 18 hours to 19 hours, 19 hours to 20 hours, 20 hours to 21 hours, 21 hours to 22 hours, 22 hours to 23 hours, 23 hours to 24 hours, 24 hours to 36 hours, or 36 hours to 48 hours. In some embodiments, the step of treating the cells with the fixative is performed at 4° C., room temperature, 40° C. or 60° C. In some embodiments, the step of treating the cells with the fixative is performed at room temperature for 24 hours.

In one aspect of the method provided herein, the paraffin embedding of fixed cells comprises: contacting the cells with ethanol; contacting the cells with xylene; and incubating the cells with paraffin.

In some embodiments, the step of contacting the cells with ethanol comprises: contacting the cells with 70% ethanol in water for 30 minutes; contacting the cells with 80% ethanol in water for 30 minutes; contacting the cells with 95% ethanol in water for 30 minutes; and contacting the cells with 100% ethanol for 30 minutes.

In some embodiments, the step of contacting the cells with xylene comprises three changes of xylene for 20 minutes each.

In some embodiments, the step of incubating the cells with paraffin comprises four changes of paraffin for 20 minutes each. In some embodiments, the step of incubating the cells with paraffin comprises is carried out at 60° C.

In some embodiments, the cells are deparaffinized by contacting the cells with xylene. In some embodiments, the cells are contacted with xylene for about 5 minutes to 10 minutes, 10 minutes to 15 minutes, 15 minutes to 20 minutes, 20 minutes to 30 minutes, 30 minutes to 60 minutes, 60 minutes to 90 minutes, or 90 minutes to 120 minutes.

In some embodiments, the cells are further contacted with successive ethanol gradients. In some embodiments, wherein the cells are further contacted with successive ethanol gradients comprising: contacting the cells with 70% ethanol in water for about 15 to about 30 minutes; contacting the cells with 95% ethanol in water for about 15 to about 30 minutes; and finally contacting the cells with 100% ethanol for about 15 to about 30 minutes.

In one aspect of the method provided herein, the cells are resuspended in antigen retrieval solution.

In some embodiments, the cells are further heated at 95° C. for about 30 minutes. In some embodiments, the cells are heated by microwave radiation.

In some embodiments, the first and/or second detection agent is an antibody or antigen binding fragment thereof. In some embodiments, the first and/or second detection agent is an RNA based binder molecule.

In one aspect of the method provided herein, the sample comprises cells from a bodily fluid or tissue. In some embodiments, the bodily fluid is blood, serum or plasma. In some embodiments, the sample is from a patient. In some embodiments, the patient is a mammal. In some embodiments, the mammal is a human. In some embodiments, the sample comprises cells from an immortalized cell line.

In another aspect, provided herein is a kit for performing the method of any one of the embodiments provided herein.

In another aspect, provided herein is a sample of cells prepared according to the method of any one of embodiments provided herein.

### BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIG. 1 shows a schematic of the method as provided herein for enhanced detection and screening of molecules.

FIG. 2 shows antibody binding in the form of optical density measured at 450 nm. Each antibody binding was tested in duplicate on 30,000 cells per well. The condition named: “Control neg” pertained to cells incubated with the antibody diluent only.

FIGS. 3A-3B show results of immunohistochemistry (“IHC”) assay performed with various antibody solutions on 4 um sections of the cell pellet blocks. FIG. 3A shows IHC cell pellet staining for each antibody. FIG. 3B shows H scores for each stained pellet. H-scores for each stained pellet was calculated based on area quantification algorithm computing the total surface intensity staining of the cell pellet.

FIG. 4 shows correlation assessment between the ELISA-like method provided herein and IHC staining. The Pearson correlation coefficient r=0.929 (GraphPad Prism) indicated adequate comparability between the screening result obtained via the high through put ELISA-like method and the traditional IHC performed on formalin fixed paraffin embedded (“FFPE”) tissue samples.

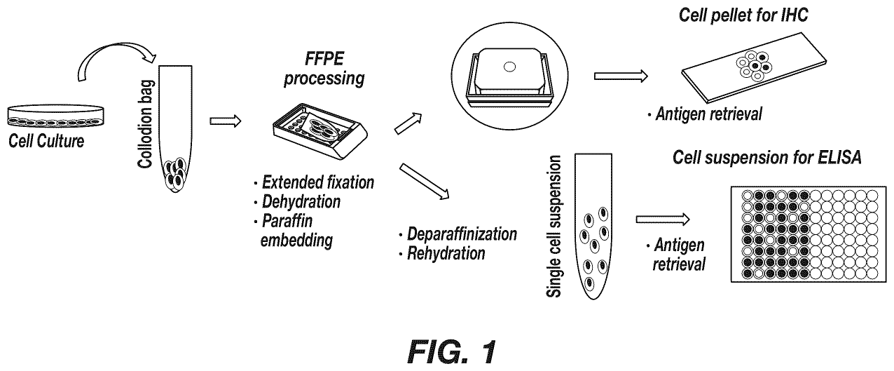

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dubel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The following references are incorporated herein by reference in their entirety: U.S. patent application Ser. No. 10/872,462, U.S. patent application Ser. No. 14/115,327, U.S. patent application Ser. No. 13/536,021, U.S. patent application Ser. No. 10/320,219, U.S. patent application Ser. No. 11/319,118, U.S. patent application Ser. No. 14/652,407, U.S. patent application Ser. No. 13/571,854, U.S. patent application Ser. No. 11/772,288, McGinnis et al. (Journal of Pathology; 2021; 254(4); 405-417), Sun et al. (PLoS ONE; 16(2) e0247238; 2021), Gentles et al. (Journal of Clinical Pathology 2021; 74:469-474), Wilgenbusch et al., 2020 (Journal of the American Society of Cytopathology; 9, 20-25) and Mairaville et al. (Antibodies; 2021, 10, 4).

### 5.1. Definitions

It is noted here that as used in this specification and the appended claims, the singular forms “a,” “an,” and “the” include plural reference unless the context clearly dictates otherwise.

In the event there is a plurality of definitions for a term herein, those in this section prevail unless stated otherwise.

The term “about” or “approximately” means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 20%, 15%, 10%, 9%, 8%, 7%, 6%, 5%, 4%, 3%, 2%, 1%, 0.5%, or 0.05% of a given value or range.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “administer” or “administration” refers to the act of injecting or otherwise physically delivering a substance as it exists outside the body into a patient, such as by oral, mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or a symptom thereof, is being prevented, administration of the substance typically occurs before the onset of the disease or symptoms thereof.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, single domain antibodies (e.g., VHH) and fragments thereof (e.g., domain antibodies). An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

“Antigen binding domain” or “antigen binding fragment” or “domain that binds an antigen” refers to a portion of a molecule that specifically binds an antigen. Antigen binding domain may include portions of an immunoglobulin that bind an antigen, such as a heavy chain variable domain (VH), a light chain variable domain (VL), the VH and the VL, Fab, Fab′, F(ab′)2, Fd and Fv fragments, domain antibodies (dAb) consisting of one VH or one VL, shark variable IgNAR domains, camelized VH domains, VHH, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3 and non-antibody scaffolds that bind an antigen.

As used herein, an “epitope” is a term in the art and refers to a localized region of an antigen to which a binding molecule (e.g., an antibody comprising a single chain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

An “intact” antibody is one comprising an antigen-binding site as well as a constant domain of the light chain (CL) and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Single domain antibody” or “sdAb” as used herein refers to a single monomeric variable antibody domain and which is capable of antigen binding. Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a functional exogenous receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (koff) to association rate (kon) of a binding molecule (e.g., an antibody) to a monovalent antigen (koff/kon) is the dissociation constant KD, which is inversely related to affinity. The lower the KD value, the higher the affinity of the antibody. The value of KD varies for different complexes of antibody and antigen and depends on both kon and koff. The dissociation constant KD for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

The term “body fluid” or “bodily fluid” as used herein refers to a fluid that is obtained from a patient, such as a mammal (e.g., human) patient. For example, a body fluid may be blood, cerebral spinal fluid (CSF), breast milk or urine. The body fluid can also be blood fractionated to remove cells (i.e., plasma) or cells and clotting factors (i.e., serum).

The term “capture moiety” or “first antibody” as used herein refers to a composition that is capable of being specifically bound by another composition that is immobilized, e.g., attached or otherwise linked, to a solid support. Many of the detection moieties provided herein can also be used as capture moieties so long as a binding event is involved. For example, useful capture moieties include affinity labels for which specific and selective ligands are available (e.g., biotin with avidin, glutathione with GST), haptens and proteins for which antisera or monoclonal antibodies are available (e.g., c-Myc), nucleic acid molecules with a sequence complementary to a target, and peptides for which specific and selective ligands are available (e.g., histidine tag with Ni). Molecules that affect the binding characteristics to a chromatographic resin are also envisioned. The solid support can be, for example, a filter, a plate, a membrane, a chromatographic resin, or a bead.

The term “cutpoint factor” or “threshold” as used herein generally refers to a value that is used to mathematically manipulate the signal from the naïve pooled matrix (e.g., serum or plasma) to set the minimum signal required from a sample to be considered positive.

The term “derivative” when used in connection with antibody substances and polypeptides used in the methods provided herein refers to polypeptides chemically modified by techniques including, but not limited to, ubiquitination, conjugation to therapeutic or diagnostic agents, labeling (e.g., with radionuclides or various enzymes), covalent polymer attachment such as pegylation (i.e., derivatization with polyethylene glycol) and insertion or substitution by chemical synthesis of amino acids such as ornithine, which do not normally occur in human proteins. Derivatives can retain the binding properties of underivatized molecules.

The terms “detectable moiety,” “detection moiety” or a “label” as used herein refers to a composition (e.g., polypeptide or antibody) detectable by means including, but not limited to, spectroscopic, photochemical, biochemical, immunochemical, chemical, or other physical means. For example, useful detectable moieties or labels include Ruthenium (Ru)-based catalyst, Europium, ³²P, ³⁵S, fluorescent dyes, electron-dense reagents, enzymes (e.g., as commonly used in an ELISA), biotin-Streptavidin, dioxigenin, haptens and proteins for which antisera or monoclonal antibodies are available, and nucleic acid molecules with a sequence complementary to a target. The detectable moiety or label often generates a measurable signal, such as a radioactive, chromogenic, luminescent, or fluorescent signal, which can be used to quantitate the amount of bound detectable moiety or label in a sample.

The term “detectable antibody” as used herein refers to any antibody that can be detected. In some embodiments, the antibody is directly labeled with a detectable moiety. In certain embodiments, the antibody is a detectable anti-Ig antibody. The term “detectable anti-Ig antibody” as used herein refers to an anti-Ig antibody that can be detected. In some embodiments, the anti-Ig antibody is directly labeled with a detectable moiety in addition to its inherent binding to an Ig molecule. The Ig antibody can be of, for example, the IgG, IgE, IgM, IgD, IgA or IgY isotype.

As used herein, the term “primary antibody” refers to an antibody that binds directly to the antigen of interest. As used herein, the term “secondary antibody” refers to an antibody that is conjugated to a detection label. In some embodiments, the secondary antibody provided herein binds directly to the primary antibody. In other embodiments, the secondary antibody provided herein binds indirectly to the primary antibody, e.g., by binding to another antibody that recognizes the primary antibody.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. Any fragment of the peptide or polypeptide disclosed herein is functional. In certain embodiments, fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of an antibody that immunospecifically binds to a target antigen. In a specific embodiment, the antibody fragment that immunospecifically binds to a target antigen, retains at least 1, at least 2, or at least 3 functions of the antibody.

The terms “identical” or percent “identity,” in the context of two or more polynucleotide or polypeptide sequences, refer to two or more sequences or subsequences that are the same or have a specified percentage of nucleotides or amino acid residues that are the same, when compared and aligned for maximum correspondence, as measured using a sequence comparison algorithms or by visual inspection.

The term “antibody that immunospecifically binds” with a target antigen and analogous terms are used interchangeably herein and refer to antibodies and fragments thereof, that specifically bind to only the target antigen or epitope. In yet other embodiments, antibodies provided herein immunospecifically bind to an Ig, such as an IgG, IgE, IgM, IgD, IgA isotype.

The term “interference” as used herein generally refers to the presence of substances in body fluid (e.g., serum or plasma) samples that prevent the target analyte from accurate detection and measurement. As used herein, interference generally refers to the effect of free drug or the effect of the matrix (e.g., serum or plasma) on the concentration-response relationship. For example, interference from matrix may be evaluated as the relative accuracy to samples without the potential interference to target a range of 75-125% relative accuracy.

The term “in vivo,” in the context of samples, refers to samples obtained from a subject, e.g., a patient, such as a human patient, including biological samples such as biological or body fluids, e.g., blood, plasma, serum, bone marrow, spinal fluid, brain fluid, or tissues, such as lymph tissue, a thin layer cytological sample, a fresh frozen tissue sample or a tumor tissue. The term “in vivo” is to be distinguished from the term “in vitro,” which encompasses cells or cell lines or biomolecular components of cells that have been cultured or propagated outside of a living organism.

The term “limit of detection,” “LOD” or “sensitivity” as used herein generally refers to the lowest analyte concentration in a body fluid (e.g., serum or plasma) sample that can be detected but not necessarily quantitated as an exact value. For example, LOD may be defined as the analyte concentration that consistently generates a signal greater than the measured mean response of the pooled naïve matrix plus a cutpoint factor.

The term “matrix” or “matrices” as used herein generally refers to the biological background in which the antibodies are measured. Examples of matrices include, for example, body fluid and tissue.

The term “monoclonal antibody” refers to an antibody obtained from a population of homogenous or substantially homogeneous antibodies, and each monoclonal antibody will typically recognize a single epitope on the antigen. In certain embodiments, a “monoclonal antibody,” as used herein, is an antibody produced by a single hybridoma or other cell, wherein the antibody immunospecifically binds to only an enzyme, as determined, e.g., by ELISA or other antigen-binding or competitive binding assay known in the art. The term “monoclonal” is not limited to any particular method for making the antibody. For example, monoclonal antibodies used in the methods provided herein may be made by the hybridoma method as described in Kohler et al.; Nature, 256:495 (1975) or may be isolated from phage libraries using the techniques known in the art. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York).

“Polyclonal antibodies” as used herein refers to an antibody population generated in an immunogenic response to a protein having many epitopes and thus includes a variety of different antibodies directed to the same and to different epitopes within the protein. Methods for producing polyclonal antibodies are known in the art (See, e.g., see, for example, Chapter 11 in: Short Protocols in Molecular Biology, (2002) 5th Ed., Ausubel et al., eds., John Wiley and Sons, New York).

The term “precision” as used herein generally refers to the variability in signal between the analysts and days. For example, precision may be evaluated as coefficient of variation, ranges of values, or using ANOVA statistics.

As used herein, the terms “prevent,” “preventing,” and “prevention” refer to the total or partial inhibition of the development, recurrence, onset or spread of a disease and/or symptom related thereto (e.g., a disease or symptom related thereto that is associated with elevated phenylalanine levels, such as PKU or cancer, in a patient), resulting from the administration of a therapy or combination of therapies provided herein.

The term “reagent stability” as used herein generally refers to the robustness of preparation and storage stability of the reagents. For example, reagent stability may be established by the conditions that still permit values to be measured within 75-125% accuracy relative to freshly prepared reagents.

The term “robustness” as used herein generally refers to the capacity of the assay to remain unaffected by small variations in method parameters and indicates reliability of the assay during normal run conditions. For example, robustness can be evaluated as the percent change of reagent concentration, reagent volume, or incubation time that still generates signal within 75-125% accuracy relative to the nominal conditions.

The term “sample” as used herein generally refers to a test fluid or tissue, e.g., taken from a patient, that can be used in the methods provided herein. In some embodiments, the sample is an in vivo sample, for example, bodily (or biological) fluid from a subject, e.g., a patient, such as a human patient. Non-limiting examples of such bodily fluids include blood (e.g., human peripheral blood (HPB)), blood lysate, serum, blood plasma, fine needle aspirate, ductal lavage, spinal fluid, brain fluid, bone marrow, ascites fluid or any combination thereof. In other embodiments, the sample is taken from a biopsy tissue such as a tumor tissue from a subject or a thin layer cytological sample of other body tissue or organ. In certain embodiments, the sample comprises a peripheral blood sample, tumor tissue or suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample. In other embodiments, the sample is an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample.

In some embodiments provided herein, the sample comprises cells from cell lines. In one embodiment, the cell lines are cell lines, such as Vero cells, CHO cells, MDCK cells, 293 T cells, HEK293T cells, Expi293F cells, BHK cells, HEK 293 cells, NS0 cells, PER.C6 cells, CRL7O3O cells, HsS78Bst cells, HeLa cells, NIH 3T3 cells or other cells lines.

In some embodiments of any one of the above- or below-mentioned embodiments, wherein the sample comprises cells from immortalized cell lines.

As used herein, “immortalized cell lines” describes a population of cells that have evaded normal cellular senescence and instead can keep undergoing division, due to a mutation(s). Cells from immortalized cell lines can therefore be grown for prolonged periods in vitro. The mutations required for immortality may occur naturally or be intentionally induced for experimental purposes. Immortalized cells may be tumorous/cancerous cells that do not stop dividing or cells that have been artificially manipulated to proliferate indefinitely and can, thus, be cultured over several generations. Without being limiting, immortalized cell lines may include: airway endothelial cells, aortic endothelial cells, Barrett's esophageal epithelial cells, bronchial epithelial cells, respiratory epithelial cells, chrondrocyte fibroblast cells, dermal microvascular endothelial cells (including TIME cells), endometrial fibroblast cells, foreskin keratinocytes, lung endothelial cells, mammary epithelial cells, mesenchymal stem cells, NTAP Schwann cells, pancreas duct cells, prostrate cells, renal epithelial cells, retinal pigmented epithelial cells and skin fibroblast cells, for example.

In some embodiments of the above- or below-mentioned embodiments, methods are provided, wherein the samples comprise cells from immortalized cell lines. Immortalized cell lines may be commercially available for uses described herein. Various immortalized cell lines are well known and commercially available (ATCC®, Manassas, VA).

The term “specificity” as used herein generally refers to the ability of the assay to detect antibodies that react with a specific protein. For example, specificity may refer to a proportional detection response with the specific analyte, while response to a non-specific protein should be below the LOD. The proportional response may be evaluated against a correlation coefficient R value greater than or equal to 0.98. When used in connection with the method provided herein to detect target antigen, specificity refers to the ability to detect antigens that react with a specific protein.

As used herein, the terms “subject” and “patient” are used interchangeably. As used herein, a subject is preferably a mammal such as a non-primate (e.g., cows, pigs, horses, cats, dogs, rats, etc.) or a primate (e.g., monkey and human), most preferably a human. In one embodiment, the subject is a mammal, preferably a human. In some embodiments of the methods and kits provided herein, the patient has a disease or symptom, or cancer. In other embodiments of the methods and kits provided herein, the patient is a patient undergoing cancer therapy. In yet other embodiments of the methods and kits provided herein, the patient is a pregnant female or an infant (e.g., age 0 to about 36 months).

As used herein, the term “tag” and “label” are used interchangeably and refer to any type of moiety that is attached to an antibody or antigen binding fragment thereof, or other polypeptide used in the methods provided herein. The term “detectable” or “detection” with reference to an antibody or tag refers to any antibody or tag that is capable of being visualized or wherein the presence of the antibody or tag is otherwise able to be determined and/or measured (e.g., by quantitation). Non-limiting examples of a detectable tag include fluorescent or other chemiluminescent tags, and tags that can be amplified and quantitated using PCR. In certain embodiments, the secondary antibody used in the methods provided herein is a biotinylated secondary antibody that is used in combination with a labeled streptavidin.

As used herein, the term “therapy” refers to any protocol, method and/or agent that can be used in the prevention, management, treatment and/or amelioration of disease (or symptom related thereto) or cancer. In certain embodiments, the terms “therapies” and “therapy” refer to a biological therapy, supportive therapy, and/or other therapies useful in the prevention, management, treatment and/or amelioration of a disease or cancer known to one of skill in the art such as medical personnel.

The term “tissue” as used herein refers to tissues that are obtained from a mammal, e.g., human. For example, a tissue may be from a biopsy sample, surgically removed tissue, or postmortem collection. Furthermore, the tissue may be homogenized and extracted to isolate the enzyme or antibodies from the tissue.

As used herein, the terms “treat,” “treatment” and “treating” refer to the reduction or amelioration of the progression, severity, and/or duration of a disease (or symptom related thereto) or cancer resulting from the administration of one or more therapies.

The term “variant” as used herein refers to a polypeptide sequence that contains at least one amino acid substitution, deletion, or insertion in the coding region relative to the original polypeptide coding domains. Variants retain the biological activity of the naturally occurring polypeptide.

As used herein, the term “in situ hybridization” or “ISH” refers to a technique for localizing and visualizing specific target nucleic acids with the preservation of morphology of the source samples.

As used herein, the term “immunohistochemistry” or “IHC” refers to a technique for detecting proteins of interest in source samples utilizing antibodies, with the preservation of morphology of the source samples. Immunofluorescence (IF) refers to fluorescent labeling, thus it is also encompassed in the term of IHC.

As used herein, the term “crosslink” refers to a process of binding two or more molecules together. The “crosslinking agent” or equivalent refers to agents containing two or more chemically reactive ends that attach themselves to the functional groups found in proteins and other molecules. Specifically, if the crosslinking agent is formaldehyde or its equivalent, a nucleophilic group on an amino acid or nucleic acid base forms a covalent bond with formaldehyde, which is stabilized in a second step that involves another functional group, often on another molecule, leading to formation of a methylene bridge. If the crosslinking agent is an oxidizing agent, it can react with the side chains of proteins and other biomolecules, allowing the formation of crosslinks that stabilize tissue structure.

As used herein, the term “fixation” or “fixing” when made in reference to fixing a sample in the IHC process refers to a procedure to preserve a sample from decay due to, e.g., autolysis or putrefaction. It terminates any ongoing biochemical reactions and may also increase the treated tissues' mechanical strength or stability.

The terms “detecting” as used herein generally refer to any form of measurement, and include determining whether an element is present or not. This term includes quantitative and/or qualitative determinations.

### 5.2 IHC

Immunohistochemistry (IHC) on formalin fixed paraffin embedded (FFPE) tissue is critical step in R&D therapeutic campaigns by identify cells expressing the target proteins of interest and predicting potential toxicities. A robust IHC assay depends on suitable primary antibodies that reliably recognize the target with optimal specificity and sensitivity. FFPE tissues often present over-fixed proteins with altered conformation, which renders repurposing of antibodies validated in non-IHC assays extremely uncertain. During the tissue preparation and preservation process, if a crosslinking agent, such as formalin, is used, formalin fixation may mask epitopes and result in decreased immunoreactivity (see Arnold et al., Biotech Histochem 71:224-230(1996)). Formalin fixation is a time-dependent process in which increased fixation time results in continued formaldehyde group binding to proteins to a point of equilibrium (see Fox et al., J Histochem Cytochem 33:845-853 (1985)). Studies have shown that formalin fixation, especially if prolonged, results in decreased antigenicity (see Battifora and Kopinski, J Histochem Cytochem 34:1095-1100(1986)), which limits the use of formalin-fixed tissues for diagnostic IHC (see Ramos-Vara, Vet Pathol 42:405-426(2005), Webster et al., J Histochem Cytochem. 57(8): 753-761(2009)). Generation of new IHC antibodies, when suitable reagents are not commercially available, requires screening many candidates against relevant controls.

The methods provided herein overcome the above-mentioned challenges by modifying the conventional protocol. Provided herein is a method that allows the screening of FFPE IHC quality antibodies using fixed cell lines (mimicking FFPE tissues) while remaining as a single-cell suspension suitable for cell-based ELISA assays. This protocol enables the high throughput testing of many antibodies and conditions, impossible to perform in a timely manner with conventional controls mounted on glass slides.

### 5.3 Methods for Enhanced Detection and Screening of Molecules

In one aspect, provided herein is a method of detecting a molecule in a sample, comprising, obtaining cells from a sample; then treating the cells with a fixative, then paraffin embedding the fixed cells, followed by deparaffinizing and suspending the cells to obtain a single cell suspension, then contacting the suspended cells with a first detection agent, then contacting the cells bound to the first detection agent with a second detection agent, and detecting the presence of the second detection agent bound to the cells of the sample.

In a preferred embodiment, the sample is obtained from a subject prior to performing the steps of the method.

In some embodiments, the first and/or second detection agent comprises an antibody or antigen binding fragment thereof, that binds at least one molecule of the suspended cells.

In some embodiments, unbound cells are removed after contacting the suspended cells with a first detection agent.

In some embodiments, the second detection agent is an antibody or fragment thereof.

In some embodiments, the unbound second detection agent is removed after contacting the cells bound to the first detection agent with a second detection agent.

In one aspect of the method provided herein, detection above background of an amount of the second detection agent bound to the sample indicates the presence of at least one molecule in the sample.

In some embodiments, before treating the cells with a fixative, the cells are cultured and pelleted in a collodion bag. In one embodiment, the collodion bag is generated by coating the inner surface of a glass conical tube with collodion solution. In one aspect, the collodion solution is first poured into a glass test tube, the solution is allowed to set for 1 hour, the collodion is poured back out while swirling the tube, the tube is dried upside down for 1 hour and then the tube is filled with tap water, covered with parafilm and stored upright in the refrigerator at 4° C. until use. In one aspect, the collodion bag is prepared as described in Wilgenbusch et al., 2020 (Journal of the American Society of Cytopathology; 9, 20-25).

In some embodiments, the molecule is a nucleic acid or a protein. In some embodiments, the nucleic acid is RNA. In some embodiments, the nucleic acid is DNA.

In one aspect, provided herein is a method of detecting a RNA in a sample, comprising, obtaining cells from a sample; then treating the cells with a fixative, then paraffin embedding the fixed cells, followed by deparaffinizing and suspending the cells to obtain a single cell suspension, then contacting the suspended cells with a first detection agent, then contacting the cells bound to the first detection agent with a second detection agent, and detecting the presence of the second detection agent bound to the cells of the sample.

In one aspect, provided herein is a method of detecting a DNA in a sample, comprising, obtaining cells from a sample; then treating the cells with a fixative, then paraffin embedding the fixed cells, followed by deparaffinizing and suspending the cells to obtain a single cell suspension, then contacting the suspended cells with a first detection agent, then contacting the cells bound to the first detection agent with a second detection agent, and detecting the presence of the second detection agent bound to the cells of the sample.

In one aspect, provided herein is a method of detecting a protein in a sample, comprising, obtaining cells from a sample; then treating the cells with a fixative, then paraffin embedding the fixed cells, followed by deparaffinizing and suspending the cells to obtain a single cell suspension, then contacting the suspended cells with a first detection agent, then contacting the cells bound to the first detection agent with a second detection agent, and detecting the presence of the second detection agent bound to the cells of the sample.

In some embodiments, the fixative is selected from a group comprising formaldehyde, paraformaldehyde, glutaraldehyde or neutral buffered formalin. In some embodiments, the fixative is formaldehyde. In some embodiments, the fixative is paraformaldehyde. In some embodiments, the fixative is glutaraldehyde. In some embodiments, the fixative is neutral buffered formalin.

In some embodiments, the method provided herein comprises treating the sample with a fixative that is a mixture of fixatives. In some embodiments, the fixative is a mixture solution of two or more fixatives, selected from a list of formaldehyde, glutaraldehyde, paraformaldehyde, neutral buffered formalin, acrolein, osmium tetroxide, permanganate fixative, dichromate fixative, and chromic acid. In one embodiment, the fixative is Bouin's fixative, which is a solution of picric acid, formaldehyde, and acetic acid. In one embodiment, the fixative is a mixture of formaldehyde and glutaraldehyde. In one embodiment, the fixative is FAA, which is a solution of ethanol, acetic acid, and formaldehyde. In one embodiment, the fixative is periodate-lysine-paraformaldehyde (PLP), which is a solution of paraformaldehyde, L-lysine, and INaO4. In one embodiment, the fixative is phosphate buffered formalin (PBF). In one embodiment, the fixative is formal calcium, which is a solution of formaldehyde and calcium chloride. In one embodiment, the fixative is formal saline, which is a solution of formaldehyde and sodium chloride. In one embodiment, the fixative is zinc formalin, which is a solution of formaldehyde and zinc sulphate. In one embodiment, the fixative is Helly's fixative, which is a solution of formaldehyde, potassium dichromate, sodium sulphate, and mercuric chloride. In one embodiment, the fixative is Hollande's fixative, which is a solution of formaldehyde, copper acetate, picric acid, and acetic acid. In one embodiment, the fixative is Gendre's solution, which is a solution of formaldehyde, ethanol, picric acid, and acetic acid glacial. In one embodiment, the fixative is alcoholic formalin, which is a solution of formaldehyde, ethanol, and calcium acetate. In one embodiment, the fixative is formol acetic alcohol, which is a solution of formaldehyde, acetic acid glacial, and ethanol. In one embodiment, the fixative is a mixture of fixatives, wherein at least one fixative of the mixture is formaldehyde, neutral buffered formalin or glutaraldehyde. In one embodiment, the fixative is fixatives that are not used at the same time but separately or consecutively, wherein at least one fixative is formaldehyde, neutral buffered formalin or glutaraldehyde.

In some embodiments, the step of treating the cells with the fixative lasts for about 30 minutes to 48 hours. In some embodiments, the step of treating the cells with the fixative lasts for about 60 minutes to 36 hours. In some embodiments, the step of treating the cells with the fixative lasts for about 2 hours to 24 hours. In some embodiments, the step of treating the cells with the fixative lasts for about 5 hours to 20 hours. In some embodiments, the step of treating the cells with the fixative lasts for about 10 hours to 15 hours. In some embodiments, the step of treating the cells with the fixative lasts for about 30 minutes to 60 minutes, 1 hour to 2 hours, 2 hours to 3 hours, 3 hours to 4 hours, 4 hours to 5 hours, 5 hours to 6 hours, 6 hours to 7 hours, 7 hours to 8 hours, 8 hours to 9 hours, 9 hours to 10 hours, 10 hours to 11 hours, 11 hours to 12 hours, 12 hours to 13 hours, 13 hours to 14 hours, 14 hours to 15 hours, 15 hours to 16 hours, 16 hours to 17 hours, 17 hours to 18 hours, 18 hours to 19 hours, 19 hours to 20 hours, 20 hours to 21 hours, 21 hours to 22 hours, 22 hours to 23 hours, 23 hours to 24 hours, 24 hours to 36 hours, or 36 hours to 48 hours. In some embodiments, the step of treating the cells with the fixative lasts for about 30 minutes to 60 minutes. In some embodiments, the step of treating the cells with the fixative lasts for about 1 hour to 2 hours. In some embodiments, the step of treating the cells with the fixative lasts for about 2 hours to 3 hours. In some embodiments, the step of treating the cells with the fixative lasts for about 3 hours to 4 hours. In some embodiments, the step of treating the cells with the fixative lasts for about 4 hours to 5 hours. In some embodiments, the step of treating the cells with the fixative lasts for about 5 hours to 6 hours. In some embodiments, the step of treating the cells with the fixative lasts for about 6 hours to 7 hours. In some embodiments, the step of treating the cells with the fixative lasts for about 7 hours to 8 hours. In some embodiments, the step of treating the cells with the fixative lasts for about 8 hours to 9 hours. In some embodiments, the step of treating the cells with the fixative lasts for about 9 hours to 10 hours. In some embodiments, the step of treating the cells with the fixative lasts for about 10 hours to 11 hours. In some embodiments, the step of treating the cells with the fixative lasts for about 11 hours to 12 hours. In some embodiments, the step of treating the cells with the fixative lasts for about 12 hours to 13 hours. In some embodiments, the step of treating the cells with the fixative lasts for about 13 hours to 14 hours. In some embodiments, the step of treating the cells with the fixative lasts for about 14 hours to 15 hours. In some embodiments, the step of treating the cells with the fixative lasts for about 15 hours to 16 hours. In some embodiments, the step of treating the cells with the fixative lasts for about 16 hours to 17 hours. In some embodiments, the step of treating the cells with the fixative lasts for about 17 hours to 18 hours. In some embodiments, the step of treating the cells with the fixative lasts for about 18 hours to 19 hours. In some embodiments, the step of treating the cells with the fixative lasts for about 19 hours to 20 hours. In some embodiments, the step of treating the cells with the fixative lasts for about 20 hours to 21 hours. In some embodiments, the step of treating the cells with the fixative lasts for about 21 hours to 22 hours. In some embodiments, the step of treating the cells with the fixative lasts for about 22 hours to 23 hours. In some embodiments, the step of treating the cells with the fixative lasts for about 23 hours to 24 hours. In some embodiments, the step of treating the cells with the fixative lasts for about 24 hours to 36 hours. In some embodiments, the step of treating the cells with the fixative lasts for about 36 hours to 48 hours.

In some embodiments, the step of treating the cells with the fixative lasts for about 30 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 36 hours, or 48 hours. In some embodiments, the step of treating the cells with the fixative lasts for about 30 minutes. In some embodiments, the step of treating the cells with the fixative lasts for about 60 minutes. In some embodiments, the step of treating the cells with the fixative lasts for about 2 hours. In some embodiments, the step of treating the cells with the fixative lasts for about 3 hours. In some embodiments, the step of treating the cells with the fixative lasts for about 4 hours. In some embodiments, the step of treating the cells with the fixative lasts for about 5 hours. In some embodiments, the step of treating the cells with the fixative lasts for about 6 hours. In some embodiments, the step of treating the cells with the fixative lasts for about 7 hours. In some embodiments, the step of treating the cells with the fixative lasts for about 8 hours. In some embodiments, the step of treating the cells with the fixative lasts for about 9 hours. In some embodiments, the step of treating the cells with the fixative lasts for about 10 hours. In some embodiments, the step of treating the cells with the fixative lasts for about 11 hours. In some embodiments, the step of treating the cells with the fixative lasts for about 12 hours. In some embodiments, the step of treating the cells with the fixative lasts for about 13 hours. In some embodiments, the step of treating the cells with the fixative lasts for about 14 hours. In some embodiments, the step of treating the cells with the fixative lasts for about 15 hours. In some embodiments, the step of treating the cells with the fixative lasts for about 16 hours. In some embodiments, the step of treating the cells with the fixative lasts for about 17 hours. In some embodiments, the step of treating the cells with the fixative lasts for about 18 hours. In some embodiments, the step of treating the cells with the fixative lasts for about 19 hours. In some embodiments, the step of treating the cells with the fixative lasts for about 20 hours. In some embodiments, the step of treating the cells with the fixative lasts for about 21 hours. In some embodiments, the step of treating the cells with the fixative lasts for about 22 hours. In some embodiments, the step of treating the cells with the fixative lasts for about 23 hours. In some embodiments, the step of treating the cells with the fixative lasts for about 24 hours. In some embodiments, the step of treating the cells with the fixative lasts for about 25 hours. In some embodiments, the step of treating the cells with the fixative lasts for about 26 hours. In some embodiments, the step of treating the cells with the fixative lasts for about 27 hours. In some embodiments, the step of treating the cells with the fixative lasts for about 28 hours. In some embodiments, the step of treating the cells with the fixative lasts for about 29 hours. In some embodiments, the step of treating the cells with the fixative lasts for about 30 hours. In some embodiments, the step of treating the cells with the fixative lasts for about 36 hours. In some embodiments, the step of treating the cells with the fixative lasts for about 48 hours.

In some embodiments, the method provided herein comprises treating the sample with a fixative at a temperature of 0° C. to 100° C. In some embodiments, the method provided herein comprises treating the sample with a fixative at a temperature of 1° C. to 90° C. In some embodiments, the method provided herein comprises treating the sample with a fixative at a temperature of 2° C. to 80° C. In some embodiments, the method provided herein comprises treating the sample with a fixative at a temperature of 3° C. to 70° C. In some embodiments, the method provided herein comprises treating the sample with a fixative at a temperature of 4° C. to 60° C. In some embodiments, the method provided herein comprises treating the sample with a fixative at a temperature of 0° C. to 10° C. In some embodiments, the method provided herein comprises treating the sample with a fixative at a temperature of 10° C. to 20° C. In some embodiments, the method provided herein comprises treating the sample with a fixative at a temperature of 20° C. to 30° C. In some embodiments, the method provided herein comprises treating the sample with a fixative at a temperature of 30° C. to 40° C. In some embodiments, the method provided herein comprises treating the sample with a fixative at a temperature of 40° C. to 50° C. In some embodiments, the method provided herein comprises treating the sample with a fixative at a temperature of 50° C. to 60° C. In some embodiments, the method provided herein comprises treating the sample with a fixative at a temperature of 60° C. to 70° C. In some embodiments, the method provided herein comprises treating the sample with a fixative at a temperature of 70° C. to 80° C. In some embodiments, the method provided herein comprises treating the sample with a fixative at a temperature of 80° C. to 90° C. In some embodiments, the method provided herein comprises treating the sample with a fixative at a temperature of 90° C. to 100° C.

In some embodiments, the step of treating the cells with the fixative is performed at 4° C., room temperature, 40° C. or 60° C. In some embodiments, the step of treating the cells with the fixative is performed at 4° C. In some embodiments, the step of treating the cells with the fixative is performed at room temperature. In some embodiments, the step of treating the cells with the fixative is performed at 40° C. In some embodiments, the step of treating the cells with the fixative is performed at 60° C.

In some embodiments, the method provided herein comprises treating the sample with 1% to 20% neutral buffered formalin. In some embodiments, the method provided herein comprises treating the sample with 1% neutral buffered formalin. In some embodiments, the method provided herein comprises treating the sample with 5% neutral buffered formalin. In some embodiments, the method provided herein comprises treating the sample with 8% neutral buffered formalin. In some embodiments, the method provided herein comprises treating the sample with 10% neutral buffered formalin. In some embodiments, the method provided herein comprises treating the sample with 12% neutral buffered formalin. In some embodiments, the method provided herein comprises treating the sample with 15% neutral buffered formalin. In some embodiments, the method provided herein comprises treating the sample with 20% neutral buffered formalin. In some embodiments, the method provided herein comprises treating the sample with 1% to 5% neutral buffered formalin. In some embodiments, the method provided herein comprises treating the sample with 5% to 10% neutral buffered formalin. In some embodiments, the method provided herein comprises treating the sample with 10% to 15% neutral buffered formalin. In some embodiments, the method provided herein comprises treating the sample with 15% to 20% neutral buffered formalin.

In some embodiments, the method provided herein comprises treating the sample with 1% to 20% neutral buffered formalin at a temperature of 0° C. to 100° C. In some embodiments, the method provided herein comprises treating the sample with 1% to 20% neutral buffered formalin at a temperature of 1° C. to 90° C. In some embodiments, the method provided herein comprises treating the sample with 1% to 20% neutral buffered formalin at a temperature of 2° C. to 80° C. In some embodiments, the method provided herein comprises treating the sample with 1% to 20% neutral buffered formalin at a temperature of 3° C. to 70° C. In some embodiments, the method provided herein comprises treating the sample with 1% to 20% neutral buffered formalin at a temperature of 4° C. to 60° C.

In some embodiments, the method provided herein comprises treating the sample with 10% neutral buffered formalin at a temperature of 0° C. to 20° C. In some embodiments, the method provided herein comprises treating the sample with 10% neutral buffered formalin at a temperature of 20° C. to 40° C. In some embodiments, the method provided herein comprises treating the sample with 10% neutral buffered formalin at a temperature of 40° C. to 60° C. In some embodiments, the method provided herein comprises treating the sample with 10% neutral buffered formalin at a temperature of 60° C. to 80° C. In some embodiments, the method provided herein comprises treating the sample with 10% neutral buffered formalin at a temperature of 80° C. to 100° C.

In some embodiments, the method provided herein comprises treating the sample with 10% neutral buffered formalin at a temperature of 0° C. to 100° C. In some embodiments, the method provided herein comprises treating the sample with 10% neutral buffered formalin at a temperature of 1° C. to 90° C. In some embodiments, the method provided herein comprises treating the sample with 10% neutral buffered formalin at a temperature of 2° C. to 80° C. In some embodiments, the method provided herein comprises treating the sample with 10% neutral buffered formalin at a temperature of 3° C. to 70° C. In some embodiments, the method provided herein comprises treating the sample with 10% neutral buffered formalin at a temperature of 4° C. to 60° C.

In some embodiments, the method provided herein comprises treating the sample with 10% neutral buffered formalin (“NBF”) at a temperature of about 1° C. In some embodiments, the method provided herein comprises treating the sample with 10% NBF at a temperature of about 2° C. In some embodiments, the method provided herein comprises treating the sample with 10% NBF at a temperature of about 3° C. In some embodiments, the method provided herein comprises treating the sample with 10% NBF at a temperature of about 4° C. In some embodiments, the method provided herein comprises treating the sample with 10% NBF at a temperature of about 5° C. In some embodiments, the method provided herein comprises treating the sample with 10% NBF at a temperature of about 6° C. In some embodiments, the method provided herein comprises treating the sample with 10% NBF at a temperature of about 7° C. In some embodiments, the method provided herein comprises treating the sample with 10% NBF at a temperature of about 8° C. In some embodiments, the method provided herein comprises treating the sample with 10% NBF at a temperature of about 9° C. In some embodiments, the method provided herein comprises treating the sample with 10% NBF at a temperature of about 10° C. In some embodiments, the method provided herein comprises treating the sample with 10% NBF at a temperature of about 11° C. In some embodiments, the method provided herein comprises treating the sample with 10% NBF at a temperature of about 12° C. In some embodiments, the method provided herein comprises treating the sample with 10% NBF at a temperature of about 13° C. In some embodiments, the method provided herein comprises treating the sample with 10% NBF at a temperature of about 14° C. In some embodiments, the method provided herein comprises treating the sample with 10% NBF at a temperature of about 15° C. In some embodiments, the method provided herein comprises treating the sample with 10% NBF at a temperature of about 16° C. In some embodiments, the method provided herein comprises treating the sample with 10% NBF at a temperature of about 17° C. In some embodiments, the method provided herein comprises treating the sample with 10% NBF at a temperature of about 18° C. In some embodiments, the method provided herein comprises treating the sample with 10% NBF at a temperature of about 19° C. In some embodiments, the method provided herein comprises treating the sample with 10% NBF at a temperature of about 20° C. In some embodiments, the method provided herein comprises treating the sample with 10% NBF at a temperature of about 21° C. In some embodiments, the method provided herein comprises treating the sample with 10% NBF at a temperature of about 22° C. In some embodiments, the method provided herein comprises treating the sample with 10% NBF at a temperature of about 23° C. In some embodiments, the method provided herein comprises treating the sample with 10% NBF at a temperature of about 24° C. In some embodiments, the method provided herein comprises treating the sample with 10% NBF at a temperature of about 25° C. In some embodiments, the method provided herein comprises treating the sample with 10% NBF at a temperature of about 26° C. In some embodiments, the method provided herein comprises treating the sample with 10% NBF at a temperature of about 27° C. In some embodiments, the method provided herein comprises treating the sample with 10% NBF at a temperature of about 28° C. In some embodiments, the method provided herein comprises treating the sample with 10% NBF at a temperature of about 29° C. In some embodiments, the method provided herein comprises treating the sample with 10% NBF at a temperature of about 30° C. In some embodiments, the method provided herein comprises treating the sample with 10% NBF at a temperature of about 35° C. In some embodiments, the method provided herein comprises treating the sample with 10% NBF at a temperature of about 40° C. In some embodiments, the method provided herein comprises treating the sample with 10% NBF at a temperature of about 45° C. In some embodiments, the method provided herein comprises treating the sample with 10% NBF at a temperature of about 50° C. In some embodiments, the method provided herein comprises treating the sample with 10% NBF at a temperature of about 55° C. In some embodiments, the method provided herein comprises treating the sample with 10% NBF at a temperature of about 60° C. In some embodiments, the method provided herein comprises treating the sample with 10% NBF at a temperature of about 65° C. In some embodiments, the method provided herein comprises treating the sample with 10% NBF at a temperature of about 70° C. In some embodiments, the method provided herein comprises treating the sample with 10% NBF at a temperature of about 75° C. In some embodiments, the method provided herein comprises treating the sample with 10% NBF at a temperature of about 80° C. In some embodiments, the method provided herein comprises treating the sample with 10% NBF at a temperature of about 85° C. In some embodiments, the method provided herein comprises treating the sample with 10% NBF at a temperature of about 90° C. In some embodiments, the method provided herein comprises treating the sample with 10% NBF at a temperature of about 95° C. In some embodiments, the method provided herein comprises treating the sample with 10% NBF with 10% NBF at a temperature of about 100° C.

In some embodiments, the method provided herein comprises treating the biological sample with 1% to 20% NBF for 0.1 hour to 48 hours. In some embodiments, the method provided herein comprises treating the biological sample with 1% to 20% NBF for 0.1 hour to 36 hours. In some embodiments, the method provided herein comprises treating the biological sample with 1% to 20% NBF for 0.1 hour to 24 hours. In some embodiments, the method provided herein comprises treating the biological sample with 1% to 20% NBF for 0.2 hour to 22 hours. In some embodiments, the method provided herein comprises treating the biological sample with 1% to 20% NBF for 0.25 hour to 20 hours. In some embodiments, the method provided herein comprises treating the biological sample with 1% to 20% NBF for 0.25 hour to 18 hours.

In some embodiments, the method provided herein comprises treating the biological sample with 10% NBF for 0.1 hour to 48 hours. In some embodiments, the method provided herein comprises treating the biological sample with 10% NBF for 0.1 hour to 36 hours. In some embodiments, the method provided herein comprises treating the biological sample with 10% NBF for 0.1 hour to 24 hours. In some embodiments, the method provided herein comprises treating the biological sample with 10% NBF for 0.2 hour to 22 hours. In some embodiments, the method provided herein comprises treating the biological sample with 10% NBF for 0.25 hour to 20 hours. In some embodiments, the method provided herein comprises treating the biological sample with 10% NBF for 0.25 hour to 18 hours.

In some embodiments, the step of treating the cells with the fixative is performed at room temperature for 24 hours. In some embodiments, the step of treating the cells with 10% NBF is performed at room temperature for 24 hours.

In some specific embodiments, the method provided herein comprises treating the biological sample with a fixative at a temperature of 4° C. for more than 10 hours. In some specific embodiments, the method provided herein comprises treating the biological sample with a fixative at a temperature of 4° C. for more than 5 hours. In some specific embodiments, the method provided herein comprises treating the biological sample with a fixative at a temperature of 4° C. for more than 1 hour. In some specific embodiments, the method provided herein comprises treating the biological sample with a fixative at a temperature of 4° C. for about 5 hours. In some specific embodiments, the method provided herein comprises treating the biological sample with a fixative at a temperature of 4° C. for about 6 hours. In some specific embodiments, the method provided herein comprises treating the biological sample with a fixative at a temperature of 4° C. for about 7 hours. In some specific embodiments, the method provided herein comprises treating the biological sample with a fixative at a temperature of 4° C. for about 8 hours. In some specific embodiments, the method provided herein comprises treating the biological sample with a fixative at a temperature of 4° C. for about 9 hours. In some specific embodiments, the method provided herein comprises treating the biological sample with a fixative at a temperature of 4° C. for about 10 hours. In some specific embodiments, the method provided herein comprises treating the biological sample with a fixative at a temperature of 4° C. for about 11 hours. In some specific embodiments, the method provided herein comprises treating the biological sample with a fixative at a temperature of 4° C. for about 12 hours.

In some specific embodiments, the method provided herein comprises treating the biological sample with a fixative at room temperature for less than 6 hours. In some specific embodiments, the method provided herein comprises treating the biological sample with a fixative at room temperature for less than 3 hours. In some specific embodiments, the method provided herein comprises treating the biological sample with a fixative at room temperature for less than 1 hour. In some specific embodiments, the method provided herein comprises treating the biological sample with a fixative at room temperature for less than 0.5 hour. In some specific embodiments, the method provided herein comprises treating the biological sample with a fixative at room temperature for about 0.1 hour. In some specific embodiments, the method provided herein comprises treating the biological sample with a fixative at room temperature for about 0.15 hour. In some specific embodiments, the method provided herein comprises treating the biological sample with a fixative at room temperature for about 0.2 hour. In some specific embodiments, the method provided herein comprises treating the biological sample with a fixative at room temperature for about 0.25 hour. In some specific embodiments, the method provided herein comprises treating the biological sample with a fixative at room temperature for about 0.3 hour. In some specific embodiments, the method provided herein comprises treating the biological sample with a fixative at room temperature for about 0.35 hour. In some specific embodiments, the method provided herein comprises treating the biological sample with a fixative at room temperature for about 0.4 hour. In some specific embodiments, the method provided herein comprises treating the biological sample with a fixative at room temperature for about 0.45 hour. In some specific embodiments, the method provided herein comprises treating the biological sample with a fixative at room temperature for about 0.5 hour. In some specific embodiments, the method provided herein comprises treating the biological sample with a fixative at room temperature for about 0.55 hour. In some specific embodiments, the method provided herein comprises treating the biological sample with a fixative at room temperature for about 0.6 hour. In some specific embodiments, the method provided herein comprises treating the biological sample with a fixative at room temperature for about 0.65 hour. In some specific embodiments, the method provided herein comprises treating the biological sample with a fixative at room temperature for about 0.7 hour. In some specific embodiments, the method provided herein comprises treating the biological sample with a fixative at room temperature for about 0.75 hour. In some specific embodiments, the method provided herein comprises treating the biological sample with a fixative at room temperature for about 0.8 hour. In some specific embodiments, the method provided herein comprises treating the biological sample with a fixative at room temperature for about 0.85 hour. In some specific embodiments, the method provided herein comprises treating the biological sample with a fixative at room temperature for about 0.9 hour. In some specific embodiments, the method provided herein comprises treating the biological sample with a fixative at room temperature for about 1 hour. In some specific embodiments, the method provided herein comprises treating the biological sample with a fixative at room temperature for about 1.5 hours. In some specific embodiments, the method provided herein comprises treating the biological sample with a fixative at room temperature for about 2 hours. In some specific embodiments, the method provided herein comprises treating the biological sample with a fixative at room temperature for about 2.5 hours. In some specific embodiments, the method provided herein comprises treating the biological sample with a fixative at room temperature for about 3 hours. In some specific embodiments, the method provided herein comprises treating the biological sample with a fixative at room temperature for about 3.5 hours. In some specific embodiments, the method provided herein comprises treating the biological sample with a fixative at room temperature for about 4 hours.

In some specific embodiments, the method provided herein comprises treating the biological sample with a fixative at a temperature of 40° C. for less than 6 hours. In some specific embodiments, the method provided herein comprises treating the biological sample with a fixative at a temperature of 40° C. for less than 3 hours. In some specific embodiments, the method provided herein comprises treating the biological sample with a fixative at a temperature of 40° C. for less than 1 hour. In some specific embodiments, the method provided herein comprises treating the biological sample with a fixative at a temperature of 40° C. for less than 0.5 hour. In some specific embodiments, the method provided herein comprises treating the biological sample with a fixative at a temperature of 40° C. for about 0.1 hour. In some specific embodiments, the method provided herein comprises treating the biological sample with a fixative at a temperature of 40° C. for about 0.25 hour. In some specific embodiments, the method provided herein comprises treating the biological sample with a fixative at a temperature of 40° C. for about 0.5 hour. In some specific embodiments, the method provided herein comprises treating the biological sample with a fixative at a temperature of 40° C. for about 0.75 hour. In some specific embodiments, the method provided herein comprises treating the biological sample with a fixative at a temperature of 40° C. for about 1 hour.

In some specific embodiments, the method provided herein comprises treating the biological sample with a fixative at a temperature of 60° C. for less than 6 hours. In some specific embodiments, the method provided herein comprises treating the biological sample with a fixative at a temperature of 60° C. for less than 3 hours. In some specific embodiments, the method provided herein comprises treating the biological sample with a fixative at a temperature of 60° C. for less than 1 hour. In some specific embodiments, the method provided herein comprises treating the biological sample with a fixative at a temperature of 60° C. for less than 0.5 hour. In some specific embodiments, the method provided herein comprises treating the biological sample with a fixative at a temperature of 60° C. for about 0.1 hour. In some specific embodiments, the method provided herein comprises treating the biological sample with a fixative at a temperature of 60° C. for about 0.25 hour. In some specific embodiments, the method provided herein comprises treating the biological sample with a fixative at a temperature of 60° C. for about 0.5 hour. In some specific embodiments, the method provided herein comprises treating the biological sample with a fixative at a temperature of 60° C. for about 0.75 hour. In some specific embodiments, the method provided herein comprises treating the biological sample with a fixative at a temperature of 60° C. for about 1 hour.

In one aspect of the method provided herein, the paraffin embedding of fixed cells comprises: contacting the cells with ethanol, then contacting the cells with xylene, and incubating the cells with paraffin.

In some embodiments, the step of contacting the cells with ethanol comprises contacting the cells with 1% to 100% ethanol. In some embodiments, the step of contacting the cells with ethanol comprises contacting the cells with 5% to 95% ethanol. In some embodiments, the step of contacting the cells with ethanol comprises contacting the cells with 10% to 90% ethanol. In some embodiments, the step of contacting the cells with ethanol comprises contacting the cells with 15% to 85% ethanol. In some embodiments, the step of contacting the cells with ethanol comprises contacting the cells with 20% to 80% ethanol. In some embodiments, the step of contacting the cells with ethanol comprises: contacting the cells with 25% to 75% ethanol. In some embodiments, the step of contacting the cells with ethanol comprises: contacting the cells with 30% to 70% ethanol. In some embodiments, the step of contacting the cells with ethanol comprises: contacting the cells with 35% to 65% ethanol. In some embodiments, the step of contacting the cells with ethanol comprises: contacting the cells with 40% to 60% ethanol.

In some embodiments, the step of contacting the cells with ethanol comprises contacting the cells with ethanol for 0.1 hour to 2 hours. In some embodiments, the step of contacting the cells with ethanol comprises contacting the cells with ethanol for 0.5 hour to 1.5 hours. In some embodiments, the step of contacting the cells with ethanol comprises contacting the cells with ethanol for 0.5 hour to 1 hour. In some embodiments, the step of contacting the cells with ethanol comprises contacting the cells with ethanol for 0.1 hour to 0.5 hour. In some embodiments, the step of contacting the cells with ethanol comprises contacting the cells with ethanol for 0.5 hour to 1 hour. In some embodiments, the step of contacting the cells with ethanol comprises contacting the cells with ethanol for 1 hour to 1.5 hours. In some embodiments, the step of contacting the cells with ethanol comprises contacting the cells with ethanol for 1.5 hours to 2 hours.

In some embodiments, the step of contacting the cells with ethanol comprises contacting the cells with ethanol for 0.1 hour. In some embodiments, the step of contacting the cells with ethanol comprises contacting the cells with ethanol for 0.2 hour. In some embodiments, the step of contacting the cells with ethanol comprises contacting the cells with ethanol for 0.3 hour. In some embodiments, the step of contacting the cells with ethanol comprises contacting the cells with ethanol for 0.4 hour. In some embodiments, the step of contacting the cells with ethanol comprises contacting the cells with ethanol for 0.5 hour. In some embodiments, the step of contacting the cells with ethanol comprises contacting the cells with ethanol for 0.6 hour. In some embodiments, the step of contacting the cells with ethanol comprises contacting the cells with ethanol for 0.7 hour. In some embodiments, the step of contacting the cells with ethanol comprises contacting the cells with ethanol for 0.8 hour. In some embodiments, the step of contacting the cells with ethanol comprises contacting the cells with ethanol for 0.9 hour. In some embodiments, the step of contacting the cells with ethanol comprises contacting the cells with ethanol for 1 hour. In some embodiments, the step of contacting the cells with ethanol comprises contacting the cells with ethanol for 1.5 hours. In some embodiments, the step of contacting the cells with ethanol comprises contacting the cells with ethanol for 2 hours.

In some embodiments, the step of contacting the cells with ethanol comprises: contacting the cells with 65%-75% ethanol, then contacting the cells with 75%-85% ethanol in water, then contacting the cells with 90%-100% ethanol in water for 30 minutes. In some embodiments, the step of contacting the cells with ethanol comprises multiple concentrations of ethanol at multiple time intervals.

In some embodiments, the step of contacting the cells with ethanol comprises: contacting the cells with 70% ethanol in water for 30 minutes, then contacting the cells with 80% ethanol in water for 30 minutes, then contacting the cells with 95% ethanol in water for 30 minutes; and finally contacting the cells with 100% ethanol for 30 minutes.

In some embodiments, the step of contacting the cells with xylene comprises two changes of xylene. In some embodiments, the step of contacting the cells with xylene comprises three changes of xylene. In some embodiments, the step of contacting the cells with xylene comprises four changes of xylene. In some embodiments, the step of contacting the cells with xylene comprises five changes of xylene.

In some embodiments, the step of contacting the cells with xylene comprises two changes of xylene for 5 minutes to 40 minutes each. In some embodiments, the step of contacting the cells with xylene comprises two changes of xylene for 5 minutes each. In some embodiments, the step of contacting the cells with xylene comprises two changes of xylene for 10 minutes each. In some embodiments, the step of contacting the cells with xylene comprises two changes of xylene for 15 minutes each. In some embodiments, the step of contacting the cells with xylene comprises two changes of xylene for 20 minutes each. In some embodiments, the step of contacting the cells with xylene comprises two changes of xylene for 25 minutes each. In some embodiments, the step of contacting the cells with xylene comprises two changes of xylene for 30 minutes each. In some embodiments, the step of contacting the cells with xylene comprises two changes of xylene for 35 minutes each. In some embodiments, the step of contacting the cells with xylene comprises two changes of xylene for 40 minutes each.

In some embodiments, the step of contacting the cells with xylene comprises three changes of xylene for 5 minutes to 40 minutes each. In some embodiments, the step of contacting the cells with xylene comprises three changes of xylene for 5 minutes each. In some embodiments, the step of contacting the cells with xylene comprises three changes of xylene for 10 minutes each. In some embodiments, the step of contacting the cells with xylene comprises three changes of xylene for 15 minutes each. In some embodiments, the step of contacting the cells with xylene comprises three changes of xylene for 20 minutes each. In some embodiments, the step of contacting the cells with xylene comprises three changes of xylene for 25 minutes each. In some embodiments, the step of contacting the cells with xylene comprises three changes of xylene for 30 minutes each. In some embodiments, the step of contacting the cells with xylene comprises three changes of xylene for 35 minutes each. In some embodiments, the step of contacting the cells with xylene comprises three changes of xylene for 40 minutes each.

In some embodiments, the step of contacting the cells with xylene comprises four changes of xylene for 5 minutes to 40 minutes each. In some embodiments, the step of contacting the cells with xylene comprises four changes of xylene for 5 minutes each. In some embodiments, the step of contacting the cells with xylene comprises four changes of xylene for 10 minutes each. In some embodiments, the step of contacting the cells with xylene comprises four changes of xylene for 15 minutes each. In some embodiments, the step of contacting the cells with xylene comprises four changes of xylene for 20 minutes each. In some embodiments, the step of contacting the cells with xylene comprises four changes of xylene for 25 minutes each. In some embodiments, the step of contacting the cells with xylene comprises four changes of xylene for 30 minutes each. In some embodiments, the step of contacting the cells with xylene comprises four changes of xylene for 35 minutes each. In some embodiments, the step of contacting the cells with xylene comprises four changes of xylene for 40 minutes each.

In some embodiments, the step of contacting the cells with xylene comprises five changes of xylene for 5 minutes to 40 minutes each. In some embodiments, the step of contacting the cells with xylene comprises five changes of xylene for 5 minutes each. In some embodiments, the step of contacting the cells with xylene comprises five changes of xylene for 10 minutes each. In some embodiments, the step of contacting the cells with xylene comprises five changes of xylene for 15 minutes each. In some embodiments, the step of contacting the cells with xylene comprises five changes of xylene for 20 minutes each. In some embodiments, the step of contacting the cells with xylene comprises five changes of xylene for 25 minutes each. In some embodiments, the step of contacting the cells with xylene comprises five changes of xylene for 30 minutes each. In some embodiments, the step of contacting the cells with xylene comprises five changes of xylene for 35 minutes each. In some embodiments, the step of contacting the cells with xylene comprises five changes of xylene for 40 minutes each.

In some embodiments, the step of incubating the cells with paraffin comprises two changes of paraffin. In some embodiments, the step of incubating the cells with paraffin comprises three changes of paraffin. In some embodiments, the step of incubating the cells with paraffin comprises four changes of paraffin. In some embodiments, the step of incubating the cells with paraffin comprises five changes of paraffin.

In some embodiments, the step of incubating the cells with paraffin comprises two changes of paraffin for 5 minutes to 40 minutes each. In some embodiments, the step of incubating the cells with paraffin comprises two changes of paraffin for 5 minutes each. In some embodiments, the step of incubating the cells with paraffin comprises two changes of paraffin for 10 minutes each. In some embodiments, the step of incubating the cells with paraffin comprises two changes of paraffin for 15 minutes each. In some embodiments, the step of incubating the cells with paraffin comprises two changes of paraffin for 20 minutes each. In some embodiments, the step of incubating the cells with paraffin comprises two changes of paraffin for 25 minutes each. In some embodiments, the step of incubating the cells with paraffin comprises two changes of paraffin for 30 minutes each. In some embodiments, the step of incubating the cells with paraffin comprises two changes of paraffin for 35 minutes each. In some embodiments, the step of incubating the cells with paraffin comprises two changes of paraffin for 40 minutes each.

In some embodiments, the step of incubating the cells with paraffin comprises three changes of paraffin for 5 minutes to 40 minutes each. In some embodiments, the step of incubating the cells with paraffin comprises three changes of paraffin for 5 minutes each. In some embodiments, the step of incubating the cells with paraffin comprises three changes of paraffin for 10 minutes each. In some embodiments, the step of incubating the cells with paraffin comprises three changes of paraffin for 15 minutes each. In some embodiments, the step of incubating the cells with paraffin comprises three changes of paraffin for 20 minutes each. In some embodiments, the step of incubating the cells with paraffin comprises three changes of paraffin for 25 minutes each. In some embodiments, the step of incubating the cells with paraffin comprises three changes of paraffin for 30 minutes each. In some embodiments, the step of incubating the cells with paraffin comprises three changes of paraffin for 35 minutes each. In some embodiments, the step of incubating the cells with paraffin comprises three changes of paraffin for 40 minutes each.

In some embodiments, the step of incubating the cells with paraffin comprises four changes of paraffin for 5 minutes to 40 minutes each. In some embodiments, the step of incubating the cells with paraffin comprises four changes of paraffin for 5 minutes to 10 minutes each. In some embodiments, the step of incubating the cells with paraffin comprises four changes of paraffin for 10 minutes to 15 minutes each. In some embodiments, the step of incubating the cells with paraffin comprises four changes of paraffin for 15 minutes to 20 minutes each. In some embodiments, the step of incubating the cells with paraffin comprises four changes of paraffin for 20 minutes to 25 minutes each. In some embodiments, the step of incubating the cells with paraffin comprises four changes of paraffin for 25 minutes to 30 minutes each. In some embodiments, the step of incubating the cells with paraffin comprises four changes of paraffin for 30 minutes to 35 minutes each. In some embodiments, the step of incubating the cells with paraffin comprises four changes of paraffin for 35 minutes to 40 minutes each.

In some embodiments, the step of incubating the cells with paraffin comprises four changes of paraffin for 5 minutes each. In some embodiments, the step of incubating the cells with paraffin comprises four changes of paraffin for 10 minutes each. In some embodiments, the step of incubating the cells with paraffin comprises four changes of paraffin for 15 minutes each. In some embodiments, the step of incubating the cells with paraffin comprises four changes of paraffin for 20 minutes each. In some embodiments, the step of incubating the cells with paraffin comprises four changes of paraffin for 25 minutes each. In some embodiments, the step of incubating the cells with paraffin comprises four changes of paraffin for 30 minutes each. In some embodiments, the step of incubating the cells with paraffin comprises four changes of paraffin for 35 minutes each. In some embodiments, the step of incubating the cells with paraffin comprises four changes of paraffin for 40 minutes each.

In some embodiments, the step of incubating the cells with paraffin comprises five changes of paraffin for 5 minutes to 40 minutes each. In some embodiments, the step of incubating the cells with paraffin comprises five changes of paraffin for 5 minutes each. In some embodiments, the step of incubating the cells with paraffin comprises five changes of paraffin for 10 minutes each. In some embodiments, the step of incubating the cells with paraffin comprises five changes of paraffin for 15 minutes each. In some embodiments, the step of incubating the cells with paraffin comprises five changes of paraffin for 20 minutes each. In some embodiments, the step of incubating the cells with paraffin comprises five changes of paraffin for 25 minutes each. In some embodiments, the step of incubating the cells with paraffin comprises five changes of paraffin for 30 minutes each. In some embodiments, the step of incubating the cells with paraffin comprises five changes of paraffin for 35 minutes each. In some embodiments, the step of incubating the cells with paraffin comprises five changes of paraffin for 40 minutes each.

In some embodiments, the step of incubating the cells with paraffin is carried out between 5° C. to 100° C. In some embodiments, the step of incubating the cells with paraffin is carried out between 10° C. to 90° C. In some embodiments, the step of incubating the cells with paraffin is carried out between 20° C. to 80° C. In some embodiments, the step of incubating the cells with paraffin is carried out between 30° C. to 70° C. In some embodiments, the step of incubating the cells with paraffin is carried out between 40° C. to 60° C. In some embodiments, the step of incubating the cells with paraffin is carried out between 0° C. to 10° C. In some embodiments, the step of incubating the cells with paraffin is carried out between 10° C. to 20° C. In some embodiments, the step of incubating the cells with paraffin is carried out between 20° C. to 30° C. In some embodiments, the step of incubating the cells with paraffin is carried out between 30° C. to 40° C. In some embodiments, the step of incubating the cells with paraffin is carried out between 40° C. to 50° C. In some embodiments, the step of incubating the cells with paraffin is carried out between 50° C. to 60° C. In some embodiments, the step of incubating the cells with paraffin is carried out between 60° C. to 70° C. In some embodiments, the step of incubating the cells with paraffin is carried out between 70° C. to 80° C. In some embodiments, the step of incubating the cells with paraffin is carried out between 80° C. to 90° C. In some embodiments, the step of incubating the cells with paraffin is carried out between 90° C. to 100° C.

In some embodiments, the step of incubating the cells with paraffin is carried out at 5° C. In some embodiments, the step of incubating the cells with paraffin is carried out at 10° C. In some embodiments, the step of incubating the cells with paraffin is carried out at 15° C. In some embodiments, the step of incubating the cells with paraffin is carried out at 20° C. In some embodiments, the step of incubating the cells with paraffin is carried out at 25° C. In some embodiments, the step of incubating the cells with paraffin is carried out at 30° C. In some embodiments, the step of incubating the cells with paraffin is carried out at 35° C. In some embodiments, the step of incubating the cells with paraffin is carried out at 40° C. In some embodiments, the step of incubating the cells with paraffin is carried out at 45° C. In some embodiments, the step of incubating the cells with paraffin is carried out at 50° C. In some embodiments, the step of incubating the cells with paraffin is carried out at 55° C. In some embodiments, the step of incubating the cells with paraffin is carried out at 60° C. In some embodiments, the step of incubating the cells with paraffin is carried out at 65° C. In some embodiments, the step of incubating the cells with paraffin is carried out at 70° C. In some embodiments, the step of incubating the cells with paraffin is carried out at 75° C. In some embodiments, the step of incubating the cells with paraffin is carried out at 80° C. In some embodiments, the step of incubating the cells with paraffin is carried out at 85° C. In some embodiments, the step of incubating the cells with paraffin is carried out at 90° C. In some embodiments, the step of incubating the cells with paraffin is carried out at 95° C. In some embodiments, the step of incubating the cells with paraffin is carried out at 100° C.

In some embodiments, the cells are deparaffinized by contacting the cells with xylene. In some embodiments, the cells are contacted with xylene for about 5 minutes to 120 minutes. In some embodiments, the cells are contacted with xylene for about 5 minutes to 10 minutes, 10 minutes to 15 minutes, 15 minutes to 20 minutes, 20 minutes to 30 minutes, 30 minutes to 60 minutes, 60 minutes to 90 minutes, or 90 minutes to 120 minutes. In some embodiments, the cells are contacted with xylene for about 5 minutes. In some embodiments, the cells are contacted with xylene for about 10 minutes. In some embodiments, the cells are contacted with xylene for about 15 minutes. In some embodiments, the cells are contacted with xylene for about 20 minutes. In some embodiments, the cells are contacted with xylene for about 25 minutes. In some embodiments, the cells are contacted with xylene for about 30 minutes. In some embodiments, the cells are contacted with xylene for about 35 minutes. In some embodiments, the cells are contacted with xylene for about 40 minutes. In some embodiments, the cells are contacted with xylene for about 45 minutes. In some embodiments, the cells are contacted with xylene for about 50 minutes. In some embodiments, the cells are contacted with xylene for about 55 minutes. In some embodiments, the cells are contacted with xylene for about 60 minutes. In some embodiments, the cells are contacted with xylene for about 90 minutes. In some embodiments, the cells are contacted with xylene for about 120 minutes.

In some embodiments, the cells are further contacted with successive ethanol gradients as described above.

In one aspect of the method provided herein, the cells are resuspended in antigen retrieval solution.

In some embodiments, the cells are further heated between 60° C. to 100° C. In some embodiments, the cells are further heated between 60° C. to 70° C. In some embodiments, the cells are further heated between 70° C. to 80° C. In some embodiments, the cells are further heated between 80° C. to 90° C. In some embodiments, the cells are further heated between 90° C. to 100° C. In some embodiments, the cells are further heated at 60° C. In some embodiments, the cells are further heated at 65° C. In some embodiments, the cells are further heated at 70° C. In some embodiments, the cells are further heated at 75° C. In some embodiments, the cells are further heated at 80° C. In some embodiments, the cells are further heated at 85° C. In some embodiments, the cells are further heated at 90° C. In some embodiments, the cells are further heated at 95° C. In some embodiments, the cells are further heated at 100° C.

In some embodiments, the cells are further heated at 60° C. for about 10 minutes to 60 minutes. In some embodiments, the cells are further heated at 60° C. for about 10 minutes to 20 minutes. In some embodiments, the cells are further heated at 60° C. for about 20 minutes to 30 minutes. In some embodiments, the cells are further heated at 60° C. for about 30 minutes to 40 minutes. In some embodiments, the cells are further heated at 60° C. for about 40 minutes to 50 minutes. In some embodiments, the cells are further heated at 60° C. for about 50 minutes to 60 minutes. In some embodiments, the cells are further heated at 60° C. for about 10 minutes. In some embodiments, the cells are further heated at 60° C. for about 20 minutes. In some embodiments, the cells are further heated at 60° C. for about 30 minutes. In some embodiments, the cells are further heated at 60° C. for about 40 minutes. In some embodiments, the cells are further heated at 60° C. for about 50 minutes. In some embodiments, the cells are further heated at 60° C. for about 60 minutes.

In some embodiments, the cells are further heated at 65° C. for about 10 minutes to 60 minutes. In some embodiments, the cells are further heated at 65° C. for about 10 minutes to 20 minutes. In some embodiments, the cells are further heated at 65° C. for about 20 minutes to 30 minutes. In some embodiments, the cells are further heated at 65° C. for about 30 minutes to 40 minutes. In some embodiments, the cells are further heated at 65° C. for about 40 minutes to 50 minutes. In some embodiments, the cells are further heated at 65° C. for about 50 minutes to 60 minutes. In some embodiments, the cells are further heated at 65° C. for about 10 minutes. In some embodiments, the cells are further heated at 65° C. for about 20 minutes. In some embodiments, the cells are further heated at 65° C. for about 30 minutes. In some embodiments, the cells are further heated at 65° C. for about 40 minutes. In some embodiments, the cells are further heated at 65° C. for about 50 minutes. In some embodiments, the cells are further heated at 65° C. for about 60 minutes.

In some embodiments, the cells are further heated at 70° C. for about 10 minutes to 60 minutes. In some embodiments, the cells are further heated at 70° C. for about 10 minutes to 20 minutes. In some embodiments, the cells are further heated at 70° C. for about 20 minutes to 30 minutes. In some embodiments, the cells are further heated at 70° C. for about 30 minutes to 40 minutes. In some embodiments, the cells are further heated at 70° C. for about 40 minutes to 50 minutes. In some embodiments, the cells are further heated at 70° C. for about 50 minutes to 60 minutes. In some embodiments, the cells are further heated at 70° C. for about 10 minutes. In some embodiments, the cells are further heated at 70° C. for about 20 minutes. In some embodiments, the cells are further heated at 70° C. for about 30 minutes. In some embodiments, the cells are further heated at 70° C. for about 40 minutes. In some embodiments, the cells are further heated at 70° C. for about 50 minutes. In some embodiments, the cells are further heated at 70° C. for about 60 minutes.

In some embodiments, the cells are further heated at 80° C. for about 10 minutes to 60 minutes. In some embodiments, the cells are further heated at 80° C. for about 10 minutes to 20 minutes. In some embodiments, the cells are further heated at 80° C. for about 20 minutes to 30 minutes. In some embodiments, the cells are further heated at 80° C. for about 30 minutes to 40 minutes. In some embodiments, the cells are further heated at 80° C. for about 40 minutes to 50 minutes. In some embodiments, the cells are further heated at 80° C. for about 50 minutes to 60 minutes. In some embodiments, the cells are further heated at 80° C. for about 10 minutes. In some embodiments, the cells are further heated at 80° C. for about 20 minutes. In some embodiments, the cells are further heated at 80° C. for about 30 minutes. In some embodiments, the cells are further heated at 80° C. for about 40 minutes. In some embodiments, the cells are further heated at 80° C. for about 50 minutes. In some embodiments, the cells are further heated at 80° C. for about 60 minutes.

In some embodiments, the cells are further heated at 85° C. for about 10 minutes to 60 minutes. In some embodiments, the cells are further heated at 85° C. for about 10 minutes to 20 minutes. In some embodiments, the cells are further heated at 85° C. for about 20 minutes to 30 minutes. In some embodiments, the cells are further heated at 85° C. for about 30 minutes to 40 minutes. In some embodiments, the cells are further heated at 85° C. for about 40 minutes to 50 minutes. In some embodiments, the cells are further heated at 85° C. for about 50 minutes to 60 minutes. In some embodiments, the cells are further heated at 85° C. for about 10 minutes. In some embodiments, the cells are further heated at 85° C. for about 20 minutes. In some embodiments, the cells are further heated at 85° C. for about 30 minutes. In some embodiments, the cells are further heated at 85° C. for about 40 minutes. In some embodiments, the cells are further heated at 85° C. for about 50 minutes. In some embodiments, the cells are further heated at 85° C. for about 60 minutes.

In some embodiments, the cells are further heated at 90° C. for about 10 minutes to 60 minutes. In some embodiments, the cells are further heated at 90° C. for about 10 minutes to 20 minutes. In some embodiments, the cells are further heated at 90° C. for about 20 minutes to 30 minutes. In some embodiments, the cells are further heated at 90° C. for about 30 minutes to 40 minutes. In some embodiments, the cells are further heated at 90° C. for about 40 minutes to 50 minutes. In some embodiments, the cells are further heated at 90° C. for about 50 minutes to 60 minutes. In some embodiments, the cells are further heated at 90° C. for about 10 minutes. In some embodiments, the cells are further heated at 90° C. for about 20 minutes. In some embodiments, the cells are further heated at 90° C. for about 30 minutes. In some embodiments, the cells are further heated at 90° C. for about 40 minutes. In some embodiments, the cells are further heated at 90° C. for about 50 minutes. In some embodiments, the cells are further heated at 90° C. for about 60 minutes.

In some embodiments, the cells are further heated at 95° C. for about 10 minutes to 60 minutes. In some embodiments, the cells are further heated at 95° C. for about 10 minutes to 20 minutes. In some embodiments, the cells are further heated at 95° C. for about 20 minutes to 30 minutes. In some embodiments, the cells are further heated at 95° C. for about 30 minutes to 40 minutes. In some embodiments, the cells are further heated at 95° C. for about 40 minutes to 50 minutes. In some embodiments, the cells are further heated at 95° C. for about 50 minutes to 60 minutes. In some embodiments, the cells are further heated at 95° C. for about 10 minutes. In some embodiments, the cells are further heated at 95° C. for about 20 minutes. In some embodiments, the cells are further heated at 95° C. for about 30 minutes. In some embodiments, the cells are further heated at 95° C. for about 40 minutes. In some embodiments, the cells are further heated at 95° C. for about 50 minutes. In some embodiments, the cells are further heated at 95° C. for about 60 minutes.

In some embodiments, the cells are further heated at 100° C. for about 10 minutes to 60 minutes. In some embodiments, the cells are further heated at 100° C. for about 10 minutes to 20 minutes. In some embodiments, the cells are further heated at 100° C. for about 20 minutes to 30 minutes. In some embodiments, the cells are further heated at 100° C. for about 30 minutes to 40 minutes. In some embodiments, the cells are further heated at 100° C. for about 40 minutes to 50 minutes. In some embodiments, the cells are further heated at 100° C. for about 50 minutes to 60 minutes. In some embodiments, the cells are further heated at 100° C. for about 10 minutes. In some embodiments, the cells are further heated at 100° C. for about 20 minutes. In some embodiments, the cells are further heated at 100° C. for about 30 minutes. In some embodiments, the cells are further heated at 100° C. for about 40 minutes. In some embodiments, the cells are further heated at 100° C. for about 50 minutes. In some embodiments, the cells are further heated at 100° C. for about 60 minutes.

In some embodiments, the cells are heated by microwave radiation. In some embodiments, the cells are heated in a water bath.

In one aspect of the method provided herein, the heating leads to retrieval of the antigen.

In some embodiments, the first and/or second detection agent is an antibody or antigen binding fragment thereof. In some embodiments, the first and/or second detection agent is an RNA based binder molecule. Any of a number of enzymes or non-enzyme labels can be utilized as a detection agent so long as the enzymatic activity or non-enzyme label, respectively, can be detected. The enzyme thereby produces a detectable signal, which can be utilized to detect a target molecule. Particularly useful detectable signals are chromogenic or fluorogenic signals. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods provided herein.

Various chromogenic or fluorogenic substrates to produce detectable signal are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-nitrophenyl phosphate (PNPP) for alkaline phosphatase; 1-methyl-3-indolyl-β-D-galactopyranoside and 2-methoxy-4-(2-nitrovinyl)phenyl β-D-galactopyranoside for β-galactosidase; 2-methoxy-4-(2-nitrovinyl)phenyl β-D-glucopyranoside for β-glucosidase; and the like. Exemplary fluorogenic substrates include, but are not limited to, 4-(trifluoromethyl)umbelliferyl phosphate for alkaline phosphatase; 4-methylumbelliferyl phosphate bis(2-amino-2-methyl-1,3-propanediol), 4-methylumbelliferyl phosphate bis (cyclohexylammonium) and 4-methylumbelliferyl phosphate for phosphatases; QuantaBlu™ and Quintolet for horseradish peroxidase; 4-methylumbelliferyl β-D-galactopyranoside, fluorescein di(β-D-galactopyranoside) and naphthofluorescein di-(β-D-galactopyranoside) for β-galactosidase; 3-acetylumbelliferyl β-D-glucopyranoside and 4-methylumbelliferyl-β- D-glucopyranoside for β-glucosidase; and 4-methylumbelliferyl-α- D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™ Warp Red™, Vulcan Fast Red and Ferangi Blue from Biocare (Concord CA; biocare.net/products/detection/chromogens).

Exemplary rare earth metals and metal isotopes suitable as a detectable label include, but are not limited to, lanthanide (III) isotopes such as ¹⁴¹Pr, ¹⁴²Nd, ¹⁴³Nd, ¹⁴⁴Nd, ¹⁴⁵Nd, ¹⁴⁶Nd, ¹⁴⁷Sm, ¹⁴⁸Nd, ¹⁴⁹Sm, ¹⁵⁰Nd, ¹⁵¹Eu, ¹⁵²Sm, ¹⁵³Eu, ¹⁵⁴Sm, ¹⁵⁵Gd, ¹⁵⁶Gd, ¹⁵⁸Gd, ¹⁵⁹Tb, ¹⁶⁰Gd, ¹⁶¹Dy, ¹⁶²Dy, ¹⁶³Dy, ¹⁶⁴Dy, ¹⁶⁵Ho, ¹⁶⁶Er, ¹⁶⁷Er, ¹⁶⁸Er, ¹⁶⁹Tm, ¹⁷⁰Er, ¹⁷¹yb¹⁷²yb, ¹⁷³Yb, ¹⁷⁴Yb, ¹⁷⁵Lu, and ¹⁷⁶Yb. Metal isotopes can be detected, for example, using time-of-flight mass spectrometry (TOF-MS) (for example, Fluidigm Helios and Hyperion systems, fluidigm.com/systems; South San Francisco, CA).

Biotin-avidin (or biotin-streptavidin) is a well-known signal amplification system based on the fact that the two molecules have extraordinarily high affinity to each other and that one avidin/streptavidin molecule can bind four biotin molecules. Antibodies are widely used for signal amplification in immunohistochemistry. Tyramide signal amplification (TSA) is based on the deposition of a large number of haptenized tyramide molecules by peroxidase activity. Tyramine is a phenolic compound. In the presence of small amounts of hydrogen peroxide, immobilized horseradish peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. publication 2012/0100540.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, OR); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NHS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

Well known methods such as microscopy, cytometry (e.g., mass cytometry, cytometry by time of flight (CyTOF), flow cytometry), or spectroscopy can be utilized to visualize chromogenic, fluorescent, or metal detectable signal associated with the respective target nucleic acids. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of nucleic acid targets in the same sample.

In some embodiments, the method provided herein comprises using a labeled primary antibody, thus eliminating the needs for performing other IHC steps. In specific embodiments, the primary antibody is labeled with chromogenic labels. In specific embodiments, the primary antibody is labeled with florescent labels. In specific embodiments, the primary antibody is labeled with polynucleotide(s). In specific embodiments, the primary antibody is labeled by NHS (succinimidyl) ester method. In specific embodiments, the primary antibody is labeled by isothiocyanate method. In specific embodiments, the primary antibody is labeled by carbodiimide method. In specific embodiments, the primary antibody is labeled by two-tag method (a catalyst and its substrate). In specific embodiments, the primary antibody is labeled by periodate method. The post-primary-antibody crosslinking can be adapted for use with fluorescent-based detection, combined with the Basescope™ signal amplification system (see Baker et al., Nature Communication 8:1998 (2017)), or combined with other nucleic acid detection methods using similar protocols.

In one aspect of the method provided herein, the sample comprises cells from a bodily fluid or tissue. In some embodiments, the bodily fluid is blood, serum or plasma. In one embodiment, the bodily fluid is blood. In one embodiment, the bodily fluid is serum. In one embodiment, the bodily fluid is plasma. In one embodiment, the sample is a tissue specimen or is derived from a tissue specimen. In one embodiment, the sample is a blood sample or is derived from a blood sample. In one embodiment, the sample is a cytological sample or is derived from a cytological sample. In one embodiment, the sample is cultured cells. In another embodiment, the sample is a sample containing exosomes. In some embodiments of any one of the above- or below-mentioned embodiments, the sample comprises immortalized cell lines.

Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the disclosure are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the disclosure can be used in diagnostic applications to identify the presence or absence of pathological cells based on the presence or absence of a nucleic acid target that is a biomarker indicative of a pathology.

It is understood by those skilled in the art that any of a number of suitable sample types can be used for detecting a target nucleic acid and a target protein using methods provided herein. The sample for use in the methods provided herein is generally a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions, cells, and/or exosomes isolated from an organism such as a mammal. Exemplary biological samples include, but are not limited to, a cell culture, including a cell, a primary cell culture, a cell line, a tissue, an organ, an organoid, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes.

Collection of cytological samples for analysis by methods provided herein are well known in the art (see, e.g., Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004).

For example, methods for processing samples for analysis of cervical tissue, including tissue biopsy and cytology samples, are well known in the art (see, e.g., Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract. Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)).

In some embodiments of any one of the above- or below- embodiments, the cells are comprised from immortalized cell lines.

In another aspect, provided herein is a kit for performing the method of any one of the embodiments provided herein. In another aspect, provided herein is a sample of cells prepared according to the method of any one of embodiments provided herein.

The method provided herein is a valuable research tool as well as diagnostic tool. In some embodiments, the method provided herein is used for mapping spatial organization in a complex tissue. In some specific embodiments, the method provided herein is used for identifying cell types and new cell types. In some specific embodiments, the method provided herein is used for identifying cellular states. In other specific embodiments, the method provided herein is used for identifying cell types and new cell types in a tumor microenvironment. In some specific embodiments, the method provided herein is used for identifying cellular states in a tumor microenvironment.

In some embodiments, the method provided herein can be used in automated systems. In some embodiments, the method provided herein can be used in automated processes. In some embodiments, automated systems can be used for paraffin embedding of the fixed cells. In some embodiments, automated processes can be used for paraffin embedding of the fixed cells. In some embodiments, automated systems can be used for deparaffinizing of the cells. In some embodiments, automated processes can be used for deparaffinizing of the cells. In some embodiments of the various methods provided herein, each step can independently be either manual or automated. In some embodiments of the various methods provided herein, some steps are manually conducted and other steps are automated.

In some embodiments, the method provided herein is used for detecting altered gene expression in diseased cells and tissues. In some specific embodiments, the method provided herein is used for localizing altered gene expression in specific cell types and understanding tumor heterogeneity. In some specific embodiments, the method provided herein is used for studying tumor-immune cell interactions. In some embodiments, the method provided herein is used for detecting biomarkers for cancer diagnosis and prognosis. In some embodiments, the method provided herein is used for detecting therapeutic targets for cancer treatment. In some embodiments, the method provided herein is used for facilitating the validation of novel antibodies.

### Embodiments

This invention provides the following non-limiting embodiments.

In one set of embodiments, provided are:
1. A method of detecting a molecule in a sample, comprising:
   a. obtaining cells from a sample;
   b. treating the cells with a fixative;
   c. paraffin embedding the fixed cells;
   d. deparaffinizing and suspending the cells to obtain a single cell suspension;
   e. contacting the suspended cells with a first detection agent that binds at least one molecule of the suspended cells;
   f. contacting the cells bound to the first detection agent with a second detection agent; and
   g. detecting the presence of the second detection agent bound to the cells of the sample;
wherein detection above background of an amount of the second detection agent bound to the sample indicates the presence of at least one molecule in the sample.
2. The method of embodiment 1, wherein the molecule is a nucleic acid or a protein.
3. The method of embodiment 2, wherein the nucleic acid is RNA.
4. The method of embodiment 2, wherein the nucleic acid is DNA.
5. The method of any of embodiments 1 to 4, wherein the method further removing unbound cells after contacting the suspended cells with a first detection agent.
6. The method of any one of embodiments 1 to 5, wherein the method further comprising removing the unbound second detection agent after contacting the cells bound to the first detection agent with a second detection agent.
7. The method of any of embodiments 1 to 6, wherein the fixative is selected from a group comprising formaldehyde, paraformaldehyde, glutaraldehyde or neutral buffered formalin.
8. The method of embodiment 7, wherein the fixative is neutral buffered formalin.
9. The method of embodiment 8, wherein the neutral buffered formalin is 10% neutral buffered formalin.
10. The method of any one of embodiments 1 to 9, wherein the step of treating the cells with the fixative lasts for about 30 minutes to 60 minutes, 1 hour to 2 hours, 2 hours to 3 hours, 3 hours to 4 hours, 4 hours to 5 hours, 5 hours to 6 hours, 6 hours to 7 hours, 7 hours to 8 hours, 8 hours to 9 hours, 9 hours to 10 hours, 10 hours to 11 hours, 11 hours to 12 hours, 12 hours to 13 hours, 13 hours to 14 hours, 14 hours to 15 hours, 15 hours to 16 hours, 16 hours to 17 hours, 17 hours to 18 hours, 18 hours to 19 hours, 19 hours to 20 hours, 20 hours to 21 hours, 21 hours to 22 hours, 22 hours to 23 hours, 23 hours to 24 hours, 24 hours to 36 hours, or 36 hours to 48 hours.
11. The method of any one of embodiments 1 to 10, wherein the step of treating the cells with the fixative is performed at 4° C., room temperature, 40° C. or 60° C.
12. The method of any one of embodiments 1 to 11, wherein the step of treating the cells with the fixative is performed at room temperature for 24 hours.
13. The method of any one of embodiments 1 to 12, wherein the paraffin embedding of fixed cells comprises:
   a. contacting the cells with ethanol;
   b. contacting the cells with xylene; and
   c. incubating the cells with paraffin.
14. The method of embodiment 13, wherein the step of contacting the cells with ethanol comprises:
   a. contacting the cells with 70% ethanol in water for 30 minutes;
   b. contacting the cells with 80% ethanol in water for 30 minutes;
   c. contacting the cells with 95% ethanol in water for 30 minutes; and
   d. contacting the cells with 100% ethanol for 30 minutes.
15. The method of any one of embodiments 13 or 14, wherein the step of contacting the cells with xylene comprises three changes of xylene for 20 minutes each.
16. The method of any one of embodiments 13 to 15, wherein the step of incubating the cells with paraffin comprises four changes of paraffin for 20 minutes each.
17. The method of embodiment 16, wherein the step of incubating the cells with paraffin comprises is carried out at 60° C.
18. The method of any of embodiments 1 to 17, wherein the cells are deparaffinized by contacting the cells with xylene.
19. The method of embodiment 18, wherein the cells are contacted with xylene for about about 5 minutes to 10 minutes, 10 minutes to 15 minutes, 15 minutes to 20 minutes, 20 minutes to 30 minutes, 30 minutes to 60 minutes, 60 minutes to 90 minutes, or 90 minutes to 120 minutes.
20. The method of embodiments 18 or 19, wherein the cells are further contacted with successive ethanol gradients comprising: contacting the cells with 70% ethanol in water for about 15 to about 30 minutes; contacting the cells with 95% ethanol in water for about 15 to about 30 minutes; and finally contacting the cells with 100% ethanol for about 15 to about 30 minutes.
21. The method of any one of embodiments 1 to 20, wherein the cells are resuspended in antigen retrieval solution.
22. The method of embodiment 21, wherein the cells are further heated at 95° C. for about 30 minutes.
23. The method of embodiment 22, wherein the cells are heated by microwave radiation.
24. The method of any one of embodiments 1 to 23, wherein the first and/or second detection agent is an antibody or antigen binding fragment thereof.
25. The method of any one of embodiments 1 to 23, wherein the first and/or second detection agent is an RNA based binder molecule.
26. The method of any one of embodiments 1 to 25, wherein the sample comprises cells from a bodily fluid or tissue.
27. The method of embodiment 26, wherein the bodily fluid is blood, serum or plasma.
28. The method of any one of embodiments 1 to 27, wherein the sample is from a patient.
29. The method of embodiment 28, wherein the patient is a mammal.
30. The method of embodiment 29, wherein the mammal is a human.
31. The method of any one of embodiments 1 to 30, wherein the sample comprises cells from an immortalized cell line.
32. A kit for performing the method of any one of embodiments 1 to 31.
33. A sample of cells prepared according to the method of any one of embodiments 1 to 31.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### Example 1: High Throughput Screening Method for Antibodies Fit for Tissue IHC

The antibodies tested are shown in Table 1.

TABLE 1
Antibodies.

| Antibody | Provider | Cat# | Dilution |
|---|---|---|---|
| CD13 | Abcam | ab108382 | 1/750 |
| Vimentin | Abcam | ab92547 | 1/500 |
| Ki67 | Abcam | ab16667 | 1/100 |
| Alpha-SMA | Abcam | ab32575 | 1/250 |
| Lamp2 | Invitrogen | 51-2200 | 1/800 |
| CD3 | Abcam | ab16669 | 1/25 |
| GST | Argutus Medical | AML001 | 1/1000 |
| CK19 | Abcam | ab52625 | 1/800 |
| CD8 | Abcam | ab4055 | 1/200 |

NRK52E cells (Rattus norvegicus, kidney epithelial cells) were cultured and pelleted in a collodion bag before fixation in 10% NBF for 24 hours at room temperature. The collodion bag was generated by coating the entire inner surface of a 15 mL glass conical tube with 1 solution (Macron Chemicals Cat #4560-04). Following processing, a small pellet of cells was embedded in a paraffin block and the rest the cells were removed from the collodion bag and transferred in xylene. After 3×15 minutes washes in xylene (until all visible paraffin is solubilized) the cells were hydrated trough successive ethanol gradients then pelleted. The cells were resuspended in an antigen retrieval solution and heated by microwave radiation at 95° C. for 30 minutes. After cooling down they were washed in PBS, counted, and distributed in 96 well plates (30,000/well). Plates comprising filter inserts may also be used for this step and are commercially available (Millipore, Burlington, MA). For example, the filter inserts may have 0.45 um pore diameter to prevent the loss of cells in between washes and centrifugations of the plates.

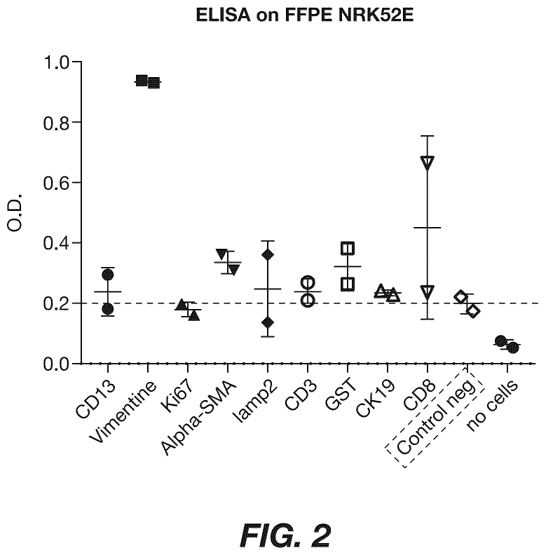

The ImmPRESS HRP Anti-Rabbit IgG Polymer Detection Kit (Vector Labs, Cat #MP-7451) was used. The cells were incubated with the antibody solutions for 60 minutes at room temperature with agitation. After washing and detection with the appropriate secondary antibodies, 50 μL of TMB solution was added per well and incubated at room temperature for 30 minutes. The development reaction was stopped, and the optical density of each well was measured at 450 nm. FIG. 2 shows antibody binding in the form of optical density measured at 450 nm. Each antibody binding was tested in duplicate on 30,000 cells per well. The condition named: “Control neg” pertained to cells incubated with the antibody diluent only.

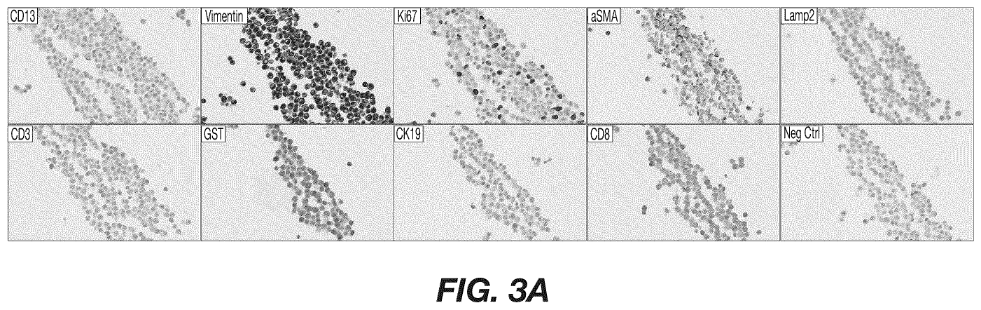

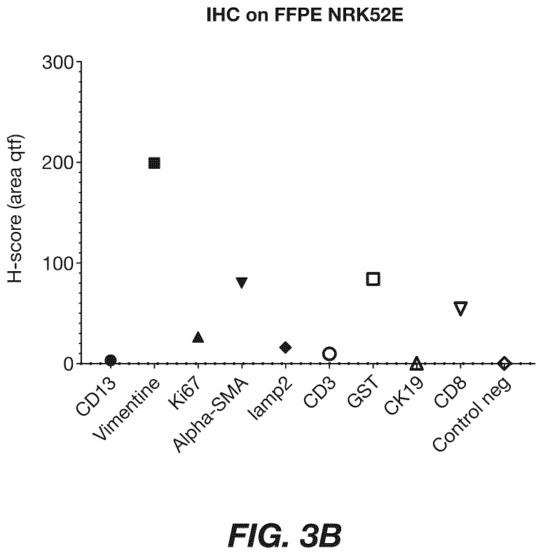

For direct comparison, an IHC assay was performed with the same antibody solutions on 4 um sections of the cell pellet blocks (IHC experiment was performed on a DAKO autostainer). H-scores for each stained pellet was calculated based on area quantification algorithm computing the total surface intensity staining of the cell pellet (Halo software, IndicaLabs). H-scores of each pellet were calculated following: H-score=(% surface area 1+)×1+(% surface area 2+)×2+(% surface area 3+)×3. FIGS. 3A-3B show results of immunohistochemistry (“IHC”) assay performed with various antibody solutions on 4 um sections of the cell pellet blocks. FIG. 3A shows IHC cell pellet staining for each antibody. FIG. 3B shows H scores for each stained pellet. H-scores for each stained pellet was calculated based on area quantification algorithm computing the total surface intensity staining of the cell pellet.

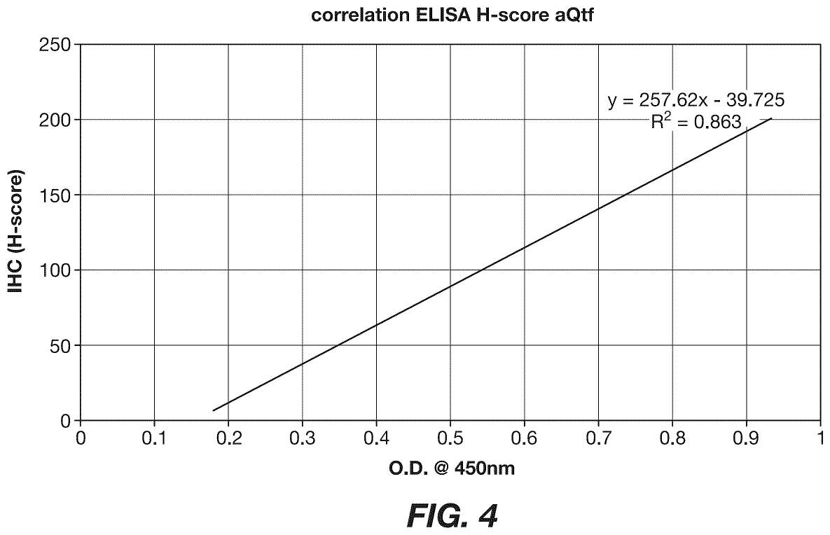

FIG. 4 shows correlation assessment between the ELISA-like method provided herein and IHC staining. The Pearson correlation coefficient r=0.929 (GraphPad Prism) indicated adequate comparability between the screening result obtained via the high through put ELISA-like method and the traditional IHC performed on formalin fixed paraffin embedded (“FFPE”) tissue samples.

### Example 2: Human Cell Materials and Methods

Human cells are obtained and cultured and pelleted in a collodion bag before fixation in 10% NBF for 24 hours at room temperature. The collodion bag is generated by coating the entire inner surface of a 15 mL glass conical tube with collodion solution (Macron Chemicals Cat #4560-04). Following processing, a small pellet of cells is embedded in a paraffin block and the rest the cells are removed from the collodion bag and transferred in xylene. After 3×15 minutes washes in xylene (until all visible paraffin is solubilized) the cells are hydrated trough successive ethanol gradients and then pelleted. The cells are resuspended in an antigen retrieval solution and heated by microwave radiation at 95° C. for 30 minutes. After cooling down they are washed in PBS, counted, and distributed in 96 well plates (30,000/well), some plates not having inserts and in some instances, plates comprising filter inserts are used, wherein the plates have 0.45 um pore diameter. Detection and quantification is performed as in Example 1.

### Example 3: Non-Human Cell Materials and Methods

Non-human cells are obtained and cultured and pelleted in a collodion bag before fixation in 10% NBF for 24 hours at room temperature. The collodion bag is generated by coating the entire inner surface of a 15 mL glass conical tube with collodion solution (Macron Chemicals Cat #4560-04). Following processing, a small pellet of cells is embedded in a paraffin block and the rest the cells are removed from the collodion bag and transferred in xylene. After 3×15 minutes washes in xylene (until all visible paraffin is solubilized) the cells are hydrated trough successive ethanol gradients then pelleted. The cells are resuspended in an antigen retrieval solution and heated in a microwave at 95° C. for 30 minutes. After cooling down they are washed in PBS, counted, and distributed in 96 well plates (30,000/well), some plates not having inserts and in some instances, plates comprising filter inserts are used, wherein the plates have 0.45 um pore diameter. Detection and quantification is performed as in Example 1.

### Example 4: Human Tissue Cell Materials and Methods

Human tissue is obtained comprising cells, and the cells from the tissues are cultured and pelleted in a collodion bag before fixation in 10% NBF for 24 hours at room temperature. The collodion bag is generated by coating the entire inner surface of a 15 mL glass conical tube with collodion solution (Macron Chemicals Cat #4560-04). Following processing, a small pellet of cells is embedded in a paraffin block and the rest the cells are removed from the collodion bag and transferred in xylene. After 3×15 minutes washes in xylene (until all visible paraffin is solubilized) the cells are hydrated trough successive ethanol gradients and then pelleted. The cells are resuspended in an antigen retrieval solution and heated by microwave radiation at 95° C. for 30 minutes. After cooling down they are washed in PBS, counted, and distributed in 96 well plates (30,000/well), some plates not having inserts and in some instances, plates comprising filter inserts are used, wherein the plates have 0.45 um pore diameter. Detection and quantification is performed as in Example 1.

### Example 5: Non-Human Tissue Cell Materials and Methods

Non-human tissue is obtained comprising cells, and the cells from the tissues are cultured and pelleted in a collodion bag before fixation in 10% NBF for 24 hours at room temperature. The collodion bag is generated by coating the entire inner surface of a 15 mL glass conical tube with collodion solution (Macron Chemicals Cat #4560-04). Following processing, a small pellet of cells is embedded in a paraffin block and the rest the cells are removed from the collodion bag and transferred in xylene. After 3×15 minutes washes in xylene (until all visible paraffin is solubilized) the cells are hydrated trough successive ethanol gradients then pelleted. The cells are resuspended in an antigen retrieval solution and heated by microwave radiation at 95° C. for 30 minutes. After cooling down they are washed in PBS, counted, and distributed in 96 well plates (30,000/well), some plates not having inserts and in some instances, plates comprising filter inserts are used, wherein the plates have 0.45 um pore diameter. Detection and quantification is performed as in Example 1.

### Example 6: Detection of Polynucleotide in Cells

Cells are obtained and cultured and pelleted in a collodion bag before fixation in 10% NBF for 24 hours at room temperature. The collodion bag is generated by coating the entire inner surface of a 15 mL glass conical tube with collodion solution (Macron Chemicals Cat #4560-04). Following processing, a small pellet of cells is embedded in a paraffin block and the rest the cells are removed from the collodion bag and transferred in xylene. After 3×15 minutes washes in xylene (until all visible paraffin is solubilized) the cells are hydrated trough successive ethanol gradients then pelleted. The cells are resuspended in an antigen retrieval solution and heated by microwave radiation at 95° C. for 30 minutes. After cooling down they are washed in PBS, counted, and distributed in 96 well plates (30,000/well), some plates not having inserts and in some instances, plates comprising filter inserts are used, wherein the plates have 0.45 um pore diameter. Detection and quantification is performed as in Example 1, wherein a polynucleotide is detected.

### Example 7: Detection of Non-Polynucleotide in Cells

Cells are obtained and cultured and pelleted in a collodion bag before fixation in 10% NBF for 24 hours at room temperature. The collodion bag is generated by coating the entire inner surface of a 15 mL glass conical tube with collodion solution (Macron Chemicals Cat #4560-04). Following processing, a small pellet of cells is embedded in a paraffin block and the rest the cells are removed from the collodion bag and transferred in xylene. After 3×15 minutes washes in xylene (until all visible paraffin is solubilized) the cells are hydrated trough successive ethanol gradients then pelleted. The cells are resuspended in an antigen retrieval solution and heated by microwave radiation at 95° C. for 30 minutes. After cooling down they are washed in PBS, counted, and distributed in 96 well plates (30,000/well), some plates not having inserts and in some instances, plates comprising filter inserts are used, wherein the plates have 0.45 um pore diameter. Detection and quantification is performed as in Example 1, wherein a molecule that is not a polypeptide is detected.

### Example 8: Immortalized Cell Materials and Methods

Immortalized cells are obtained and cultured and pelleted in a collodion bag before fixation in 10% NBF for 24 hours at room temperature. The collodion bag is generated by coating the entire inner surface of a 15 mL glass conical tube with collodion solution (Macron Chemicals Cat #4560-04). Following processing, a small pellet of cells is embedded in a paraffin block and the rest the cells are removed from the collodion bag and transferred in xylene. After 3×15 minutes washes in xylene (until all visible paraffin is solubilized) the cells are hydrated trough successive ethanol gradients then pelleted. The cells are resuspended in an antigen retrieval solution and heated by microwave radiation at 95° C. for 30 minutes. After cooling down they are washed in PBS, counted, and distributed in 96 well plates (30,000/well), some plates not having inserts and in some instances, plates comprising filter inserts are used, wherein the plates have 0.45 um pore diameter. Detection and quantification is performed as in Example 1.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

What is claimed is:

1. A method of detecting a molecule in a sample, comprising:
a. obtaining cells from a sample;
b. treating the cells with a fixative;
c. paraffin embedding the fixed cells;
d. deparaffinizing and suspending the cells to obtain a single cell suspension;
e. contacting the suspended cells with a first detection agent that binds at least one molecule of the suspended cells;
contacting the cells bound to the first detection agent with a second detection agent; and
f. detecting the presence of the second detection agent bound to the cells of the sample;
wherein detection above background of an amount of the second detection agent bound to the sample indicates the presence of at least one molecule in the sample.

2. The method of claim 1, wherein the molecule is a nucleic acid or a protein.

3. The method of claim 2, wherein the nucleic acid is RNA.

4. The method of claim 2, wherein the nucleic acid is DNA.

5. The method of any of claims 1 to 4, the method further removing unbound cells after contacting the suspended cells with a first detection agent.

6. The method of any one of claims 1-5, the method further comprising removing the unbound second detection agent after contacting the cells bound to the first detection agent with a second detection agent.

7. The method of any one of claims 1-7, wherein the fixative is selected from a group comprising formaldehyde, paraformaldehyde, glutaraldehyde or neutral buffered formalin.

8. The method of claim 7, wherein the fixative is neutral buffered formalin.

9. The method of claim 8, wherein the neutral buffered formalin is 10% neutral buffered formalin.

10. The method of any one of claims 1 to 9, wherein the step of treating the cells with the fixative lasts for about 30 minutes to 60 minutes, 1 hour to 2 hours, 2 hours to 3 hours, 3 hours to 4 hours, 4 hours to 5 hours, 5 hours to 6 hours, 6 hours to 7 hours, 7 hours to 8 hours, 8 hours to 9 hours, 9 hours to 10 hours, 10 hours to 11 hours, 11 hours to 12 hours, 12 hours to 13 hours, 13 hours to 14 hours, 14 hours to 15 hours, 15 hours to 16 hours, 16 hours to 17 hours, 17 hours to 18 hours, 18 hours to 19 hours, 19 hours to 20 hours, 20 hours to 21 hours, 21 hours to 22 hours, 22 hours to 23 hours, 23 hours to 24 hours, 24 hours to 36 hours, or 36 hours to 48 hours.

11. The method of any one of claims 1 to 10, wherein the step of treating the cells with the fixative is performed at 4° C., room temperature, 40° C. or 60° C.

12. The method of any one of claims 1 to 11, wherein the step of treating the cells with the fixative is performed at room temperature for 24 hours.

13. The method of any one of claims 1 to 12, wherein the paraffin embedding of fixed cells comprises:
a. contacting the cells with ethanol;
b. contacting the cells with xylene; and
c. incubating the cells with paraffin.

14. The method of claim 13, wherein the step of contacting the cells with ethanol comprises:
a. contacting the cells with 70% ethanol in water for 30 minutes;
b. contacting the cells with 80% ethanol in water for 30 minutes;
c. contacting the cells with 95% ethanol in water for 30 minutes; and
d. contacting the cells with 100% ethanol for 30 minutes.

15. The method of any one of claims 13 or 14, wherein the step of contacting the cells with xylene comprises three changes of xylene for 20 minutes each.

16. The method of any one of claims 13 to 15, wherein the step of incubating the cells with paraffin comprises four changes of paraffin for 20 minutes each.

17. The method of claim 16, wherein the step of incubating the cells with paraffin comprises is carried out at 60° C.

18. The method of any of claims 1 to 17, wherein the cells are deparaffinized by contacting the cells with xylene.

19. The method of claim 18, wherein the cells are contacted with xylene for about 5 minutes to 10 minutes, 10 minutes to 15 minutes, 15 minutes to 20 minutes, 20 minutes to 30 minutes, 30 minutes to 60 minutes, 60 minutes to 90 minutes, or 90 minutes to 120 minutes.

20. The method of claims 18 or 19, wherein the cells are further contacted with successive ethanol gradients comprising:
a. contacting the cells with 70% ethanol in water for about 15 to about 30 minutes;
b. contacting the cells with 95% ethanol in water for about 15 to about 30 minutes;
c. and finally contacting the cells with 100% ethanol for about 15 to about 30 minutes.

21. The method of any one of claims 1 to 20, wherein the cells are resuspended in antigen retrieval solution.

22. The method of claim 21, wherein the cells are further heated at 95° C. for about 30 minutes.

23. The method of claim 22, wherein the cells are heated by microwave radiation.

24. The method of any one of claims 1 to 23, wherein the first and/or second detection agent is an antibody or antigen binding fragment thereof.

25. The method of any one of claims 1 to 23, wherein the first and/or second detection agent is an RNA based binder molecule.

26. The method of any one of claims 1 to 25, wherein the sample comprises cells from a bodily fluid or tissue.

27. The method of claim 26, wherein the bodily fluid is blood, serum or plasma.

28. The method of any one of claims 1 to 27, wherein the sample is from a patient.

29. The method of claim 28, wherein the patient is a mammal.

30. The method of claim 29, wherein the mammal is a human.

31. The method of any one of claims 1 to 30, wherein the sample comprises cells from an immortalized cell line.

32. A kit for performing the method of any one of claims 1 to 31.

33. A sample of cells prepared according to the method of any one of claims 1 to 31.
